SEQUENCE LISTING (33 sequences)

SEQ ID NO: 1 - protein, 216 aa, Artificial Sequence
  human Angptl6 peptide
PRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMAS

SEQ ID NO: 2 - protein, 203 aa, Artificial Sequence
  mouse Angptl6 peptide
ARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSS

SEQ ID NO: 3 - DNA, 1413 bp, Homo sapiens
  mat_peptide (61)...(1410): Encodes the mature peptide
atggggaagccctggctgcgtgcgctacagctgctgctcctgctgggcgcgtcgtgggcgcgggcgggcgccccgcgctgcacctacaccttcgtgctgcccccgcagaagttcacgggcgctgtgtgctggagcggccccgcatccacgcgggcgacgcccgaggccgccaacgccagcgagctggcggcgctgcgcatgcgcgtcggccgccacgaggagctgttacgcgagctgcagaggctggcggcggccgacggcgccgtggccggcgaggtgcgcgcgctgcgcaaggagagccgcggcctgagcgcgcgcctgggccagttgcgcgcgcagctgcagcacgaggcggggcccggggcgggcccgggggcggatctgggggcggagcctgccgcggcgctggcgctgctcggggagcgcgtgctcaacgcgtccgccgaggctcagcgcgcagccgcccggttccaccagctggacgtcaagttccgcgagctggcgcagctcgtcacccagcagagcagtctcatcgcccgcctggagcgcctgtgcccgggaggcgcgggcgggcagcagcaggtcctgccgccacccccactggtgcctgtggttccggtccgtcttgtgggtagcaccagtgacaccagtaggatgctggacccagccccagagccccagagagaccagacccagagacagcaggagcccatggcttctcccatgcctgcaggtcaccctgcggtccccaccaagcctgtgggcccgtggcaggattgtgcagaggcccgccaggcaggccatgaacagagtggagtgtatgaactgcgagtgggccgtcacgtagtgtcagtatggtgtgagcagcaactggagggtggaggctggactgtgatccagcggaggcaagatggttcagtcaacttcttcactacctggcagcactataaggcgggctttgggcggccagacggagaatactggctgggccttgaacccgtgtatcagctgaccagccgtggggaccatgagctgctggttctcctggaggactgggggggccgtggagcacgtgcccactatgatggcttctccctggaacccgagagcgaccactaccgcctgcggcttggccagtaccatggtgatgctggagactctctttcctggcacaatgacaagcccttcagcaccgtggatagggaccgagactcctattctggtaactgtgccctgtaccagcggggaggctggtggtaccatgcctgtgcccactccaacctcaacggtgtgtggcaccacggcggccactaccgaagccgctaccaggatggtgtctactgggctgagtttcgtggtggggcatattctctcaggaaggccgccatgctcattcggcccctgaagctgtga

SEQ ID NO: 4 - protein, 470 aa, Homo sapiens
  DOMAIN (25)...(240): coiled-coil region
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASPMPAGHPAVPTKPVGPWQDCAEARQAGHEQSGVYELRVGRHVVSVWCEQQLEGGGWTVIQRRQDGSVNFFTTWQHYKAGFGRPDGEYWLGLEPVYQLTSRGDHELLVLLEDWGGRGARAHYDGFSLEPESDHYRLRLGQYHGDAGDSLSWHNDKPFSTVDRDRDSYSGNCALYQRGGWWYHACAHSNLNGVWHHGGHYRSRYQDGVYWAEFRGGAYSLRKAAMLIRPLKL

SEQ ID NO: 5 - DNA, 1374 bp, Mus musculus
  mat_peptide (73)...(1371): Mature peptide
atggggaccgccaggctacgcaagctgcaactgctgcttctgctgggcgcttggagggcgctcggaggtgccgcgcgttgccgcgtcaccctagttttgtccccgcagaaggcaactagcgccgtctgcaggagctcagaagccacccaagacagcgaactggccacgctgcgcatgcgcctgggtcgccacgaggagctgctgcgcgcgctgcaaaggcgtgcggcggagggtggtgcgctcgcggacgaggtgcgcgcactgcgcgagcacagtctcaccctgaacacgcgcctgggccagctgcgcgcgcaattgcagcaggaggcgagggcggagcctgacctgggggcggagcctgctgctgcacttggtttgctagccgagcgcgcgctggacgctgaggccgaagcgcgccggacgacggcacgcctgcagcagctggacgcacagctccgtgagcatgcgcagctcatgagccagcatagcagcctcctcggccgcctgcaacgcgcgtgcgcgggcccggaacggggacagcagcaggtcctgccactgcccctggcgcctctggtgcctctgagcctcgtgggcagtgccagcaacaccagcaggaggctggaccaaactccagagcaccagagagagcagagcttgagacagcaggggcctccatcttctctgctgcccacagggcaccttgctgtccccacaaggccagtgggcccatggagggattgtgcagaggctcacggggcaggtcactggcagagtggagtgtatgacctgcggctgggccgtcgtgtagtagccgtgtggtgtgaacagcagcaggaaggtggaggctggactgtcatccagagacggcaggacggctctgtcaacttcttcaccaactggcagcactacaaggcgggctttgggcgtccagaaggagaatactggctgggcctggaacctgtgcatcaggtgacaagccgtggggaccacgagctgctgatactcctagaggactgggggggccgtgcagcacgcgcccactacgacagcttctccttggagcctgagagtgaccactaccgtctgcggcttggccagtaccacggcgatgccggagactccctctcttggcacaatgacaaacctttcagcactgtggatagggacagagactcatattctggtaactgtgccctgtaccatcgtgggggctggtggtaccatgcctgtgcccactctaacctcaatggagtatggtatcatggaggtcattaccggagccgataccaggacggggtctactgggccgagttccgtggtggggcgtactctctgaagaaagctgttatgttgacccggcttgtgcgcttgtga

SEQ ID NO: 6 - protein, 457 aa, Mus musculus
  DOMAIN (24)...(227): Coiled-coil region
MGTARLRKLQLLLLLGAWRALGGAARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSSLLPTGHLAVPTRPVGPWRDCAEAHGAGHWQSGVYDLRLGRRVVAVWCEQQQEGGGWTVIQRRQDGSVNFFTNWQHYKAGFGRPEGEYWLGLEPVHQVTSRGDHELLILLEDWGGRAARAHYDSFSLEPESDHYRLRLGQYHGDAGDSLSWHNDKPFSTVDRDRDSYSGNCALYHRGGWWYHACAHSNLNGVWYHGGHYRSRYQDGVYWAEFRGGAYSLKKAVMLTRLVRL

SEQ ID NO: 7 - DNA, 675 bp, Artificial Sequence
  Encodes mouse Angptl6 with N-terminal His tag
atgggcagcagccatcatcatcatcatcacagcagcggcctggtgccgcgcggcagccatatggcgcgttgccgcgtcaccctagttttgtccccgcagaaggcaactagcgccgtctgcaggagctcagaggccacccaagacagcgaactggccacgctgcgcatgcgcctgggtcgccacgaggagctgctgcgcgcgctgcaaaggcgtgcggcggagggtggtgcgctcgcggacgaggtgcgcgcactgcgcgagcacagtctcaccctgaacacgcgcctgggccagctgcgcgcgcaattgcagcaggaggcgagggcggagcctgacctgggggcggagcctgctgctgcacttggtttgctagccgagcgcgcgctggacgctgaggccgaagcgcgccggacgacggcacgcctgcagcagctggacgcacagctccgtgagcatgcgcagctcatgagccagcatagcagcctcctcggccgcctgcaacgcgcgtgcgcgggcccggaacggggacagcagcaggtcctgccactgcccctggcgcctctggtgcctctgagcctcgtgggcagtgccagcaacaccagcaggaggctggaccaaactccagagcaccagagagagcagagcttgagacagcaggggcctccatcttcttga

SEQ ID NO: 8 - DNA, 32 bp, Artificial Sequence
  PCR primer
gagatatacatatggcgcgttgccgcgtcacc

SEQ ID NO: 9 - DNA, 34 bp, Artificial Sequence
  PCR primer
ggtgctcgagtcacaagcgcacaagccgggtcaa

SEQ ID NO: 10 - protein, 224 aa, Artificial Sequence
  Mouse Angptl6 peptide with N-terminus His tag
MGSSHHHHHHSSGLVPRGSHMARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSS

SEQ ID NO: 11 - DNA, 29 bp, Artificial Sequence
  PCR primer
tcaggatccgtgggattgccgcaaacctc

SEQ ID NO: 12 - DNA, 20 bp, Artificial Sequence
  PCR primer
agctgaaggagataggaaca

SEQ ID NO: 13 - DNA, 29 bp, Artificial Sequence
  PCR primer
tcaggatccgtgggattgccgcaaacctc

SEQ ID NO: 14 - DNA, 34 bp, Artificial Sequence
  PCR primer
ggtgctcgagtcaagaagatggaggcccctgctg

SEQ ID NO: 15 - DNA, 714 bp, Artificial Sequence
  Encodes human Angptl6 peptide with N-terminal His tag
atgggcagcagccatcatcatcatcatcacagcagcggcctggtgccgcgcggcagccatatgccgcgctgcacctacaccttcgtgctgcccccgcagaagttcacgggcgctgtgtgctggagcggccccgcatccacgcgggcgacgcccgaggccgccaacgccagcgagctggcggcgctgcgcatgcgcgtcggcagacacgaggagctgttacgcgagctgcagaggctggcggcggcggacggcgccgtggccggcgaggtgcgcgcgctgcgcaaggagagccgcggcctgagcgcgcgcctgggccagttgcgcgcgcagctgcagcacgaggcggggcccggggcgggcccgggggcggatctgggggcggagcctgccgcggcgctggcgctgctcggggagcgcgtgctcaacgcgtccgccgaggctcagcgcgcagccgcccggttccaccagctggacgtcaagttccgcgagctggcgcagctcgtcacccagcagagcagtctcatcgcccgcctggagcgcctgtgcccgggaggcgcgggcgggcagcagcaggtcctgccgccacccccactggtgcctgtggttccggtccgtcttgtgggtagcaccagtgacaccagtaggatgctggacccagccccagagccccagagagaccagacccagagacagcaggagcccatggcttcttga

SEQ ID NO: 16 - protein, 237 aa, Artificial Sequence
  Human Angptl6 peptide with N-terminal His tag
MGSSHHHHHHSSGLVPRGSHMPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMAS

SEQ ID NO: 17 - protein, 12 aa, Artificial Sequence
  Linker or hinge
ERKCCVECPPCP

SEQ ID NO: 18 - protein, 7 aa, Artificial Sequence
  Linker or hinge
VECPPCP

SEQ ID NO: 19 - protein, 15 aa, Artificial Sequence
  Linker or hinge
GGGERKCCVECPPCP

SEQ ID NO: 20 - protein, 10 aa, Artificial Sequence
  Linker or hinge
GGGVECPPCP

SEQ ID NO: 21 - protein, 444 aa, Artificial Sequence
  Mouse N-terminal Angptl6 Mouse IgG1 FC Long Hinge Mouse IgG Leader
MEWSWVFLFFLSVTTGVHSARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 22 - protein, 454 aa, Artificial Sequence
  Mouse N-terminal Angptl6 Mouse IgG1 FC Long Hinge
MGTARLRKLQLLLLLGAWRALGGAARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 23 - protein, 431 aa, Artificial Sequence
  Mouse N-terminal Angptl6 204 Mouse IgG1 FC Long Hinge
MGTARLRKLQLLLLLGAWRALGGAARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 24 - protein, 467 aa, Artificial Sequence
  Human N-terminal Angptl6 Mouse IgG1 FC Long Hinge
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 25 - protein, 468 aa, Artificial Sequence
  Human N-terminal Angptl6 Human IgG2M4 FC Long Hinge
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 463 aa, Artificial Sequence
  Human N-terminal Angptl6 Human IgG2M4 FC Short Hinge
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 471 aa, Artificial Sequence
  Human N-terminal Angptl6 Human IgG2M4 GGG FC Long Hinge
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASGGGERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 466 aa, Artificial Sequence
  Human N-terminal Angptl6 Human IgG2M4 GGG FC Short Hinge
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASGGGVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 445 aa, Artificial Sequence
  Human N-terminal Angptl6 217 Human IgG2M4 FC Long Hinge
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 239 aa, Artificial Sequence
  Mouse N-terminal Angptl6 227 6His
MEWSWVFLFFLSVTTGVHSEEFDYKDDDDKARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSSHHHHHH

SEQ ID NO: 31 - protein, 469 aa, Artificial Sequence
  Mouse Full-length Angptl6 457 6His
MEWSWVFLFFLSVTTGVHSEEFDYKDDDDKARCRVTLVLSPQKATSAVCRSSEATQDSELATLRMRLGRHEELLRALQRRAAEGGALADEVRALREHSLTLNTRLGQLRAQLQQEARAEPDLGAEPAAALGLLAERALDAEAEARRTTARLQQLDAQLREHAQLMSQHSSLLGRLQRACAGPERGQQQVLPLPLAPLVPLSLVGSASNTSRRLDQTPEHQREQSLRQQGPPSSLLPTGHLAVPTRPVGPWRDCAEAHGAGHWQSGVYDLRLGRRVVAVWCEQQQEGGGWTVIQRRQDGSVNFFTNWQHYKAGFGRPEGEYWLGLEPVHQVTSRGDHELLILLEDWGGRAARAHYDSFSLEPESDHYRLRLGQYHGDAGDSLSWHNDKPFSTVDRDRDSYSGNCALYHRGGWWYHACAHSNLNGVWYHGGHYRSRYQDGVYWAEFRGGAYSLKKAVMLTRLVRLHHHHHH

SEQ ID NO: 32 - protein, 482 aa, Artificial Sequence
  Human Full-length Angptl6 6His
MEWSWVFLFFLSVTTGVHSEEFDYKDDDDKPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASPMPAGHPAVPTKPVGPWQDCAEARQAGHEQSGVYELRVGRHVVSVWCEQQLEGGGWTVIQRRQDGSVNFFTTWQHYKAGFGRPDGEYWLGLEPVYQLTSRGDHELLVLLEDWGGRGARAHYDGFSLEPESDHYRLRLGQYHGDAGDSLSWHNDKPFSTVDRDRDSYSGNCALYQRGGWWYHACAHSNLNGVWHHGGHYRSRYQDGVYWAEFRGGAYSLRKAAMLIRPLKLHHHHHH

SEQ ID NO: 33 - protein, 193 aa, Artificial Sequence
  Human Angptl6 peptide short
PRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTS

(19) United States
(12) Patent Application Publication
(10) Pub. No.: US 2009/0104210 A1
(43) Pub. Date: Apr. 23, 2009

(54) Peptide compounds for treating obesity and insulin resistance

(76) Inventors: Michael R. Tota, Middletown, NJ (US); Shirly Pinto, Short Hills, NJ (US); Douglas J. MacNeil, Westfield, NJ (US); Heather H. Zhou, Watchung, NJ (US); Fubao Wang, Dresher, PA (US); Chen-Ni Chin, Philadelphia, PA (US)

Correspondence Address: MERCK AND CO., INC, P O BOX 2000, RAHWAY, NJ, 07065-0907, US

(21) Appl. No.: 12/287,211

(22) Filed: Oct. 7, 2008

Related U.S. Application Data
(60) Provisional application No. 60/999,274, filed on Oct. 17, 2007.

(51) Int. Cl. A61K 39/44; C07K 14/47; C07K 14/76; C07K 19/00; C07K 14/79; A61K 38/17

(52) U.S. Cl. 424/178.1; 530/350; 530/362; 530/391.7; 530/394; 514/12

(57) ABSTRACT

Compounds comprising an angiopoietin-like protein 6 (Angptl6) peptide for use in the treatment of metabolic syndrome, in particular, obesity and insulin resistance are described.

### BACKGROUND OF THE INVENTION

(1) Field of the Invention

The present invention relates to an angiopoietin-like protein 6 (Angptl6) peptides for use in the treatment of metabolic syndrome, in particular, obesity and insulin resistance.

(2) Description of Related Art

Metabolic Syndrome is a disorder that a combination of medical disorders that increase one's risk for cardiovascular disease, stroke, and diabetes and includes obesity, dyslipidaemia, and hyperglycemia. Metabolic syndrome, which is also known as (metabolic) syndrome X, insulin resistance syndrome, Reaven's syndrome, and CHAOS (Australia), has increased to epidemic proportions worldwide. The pathophysiology of this syndrome is attributed to central distributed obesity, decreased high density lipoprotein, elevated triglycerides, elevated blood pressure and hyperglycemia. People suffering from Metabolic Syndrome are at increased risk of type II diabetes, coronary heart disease, and other diseases related to plaque accumulation in artery walls (e.g., stroke and peripheral vascular disease). In two prospective European studies, Metabolic Syndrome was a predictor of increased cardiovascular disease and mortality (Isomaa et al., Diabetes Care 24: 683-689 (2001); Lakka et al., JAMA 288: 2709-2716 (2002)).

The most significant underlying cause of Metabolic Syndrome appears to be obesity. The genetic factors that also contribute to Metabolic Syndrome are not yet understood. Consequently, there is a need to identify genes that contribute to the development of Metabolic Syndrome. There is also a need for methods that permit the identification of chemical agents that modulate the activity of these genes or modulate the activity of the products (e.g., proteins) encoded by these genes. Such chemical agents may be useful, for example, as drugs to prevent Metabolic Syndrome or to ameliorate at least one symptom of Metabolic Syndrome.

WO2005097171 and Oike et al., Nat. Med. 11: 400-408 (2005) showed that a full-length Angptl6 protein antagonized obesity and insulin resistance and suggested its use as an antiobesity agent. However, full-length Angptl6 protein also caused angiogenesis, an unacceptable effect for an antiobesity treatment. Therefore, there is a need for Angptl6 protein analogs or derivatives that antagonize obesity and insulin resistance but without the undesirable angiogenesis side effects.

### BRIEF SUMMARY OF THE INVENTION

The present invention provides angiopoietin-like protein 6 (Angptl6) peptide compounds and compositions thereof that can be used therapeutically for treatment of metabolic disorders such as metabolic syndrome, in particular, reduce obesity and insulin resistance.

Therapeutic applications of the Angptl6 peptide compounds include administering the Angptl6 peptides to an individual to treat a metabolic disorder afflicting the individual. Such disorders include, but are not limited to, obesity, metabolic syndrome or syndrome X, and type II diabetes. Complications of diabetes such as retinopathy may be positively affected thereby as well. Obesity is a comorbidity of and may well contribute to such disease states as diabetes, hypertension, dyslipidemias, cardiovascular disease, gallstones, osteoarthritis and certain forms of cancers. Administration of one or more of the Angtl6 peptide compounds disclosed herein to effect weight loss in an individual may also be useful in preventing such diseases and as part of therapy for any one of the above-recited conditions, as well as others. In other embodiments, there is provided a method for treating a metabolic disease in an individual comprising administering to the individual one or more of the Angtl6 peptide compounds described above. The metabolic disease may be selected from the group consisting of diabetes, metabolic syndrome, hyperglycemia, and obesity and may be administered via a route peripheral to the brain, such as an oral, mucosal, buccal, sublingual, nasal, rectal, subcutaneous, transdermal, intravenous, intramuscular, or intraperitoneal route. Finally, the Angtl6 peptide compound can be administered to an individual to effect a reduction in food intake by the individual, to effect a reduction in weight gain in the individual, to prevent weight gain in the individual, to effect weight loss in the individual, and/or to prevent weight regain in the individual.

Accordingly, the present invention provides Angptl6 peptide compounds comprising the coiled-coil domain of an Angptl6 proteins and excluding an intact globular fibrinogen domain of the Angptl6 protein and compositions thereof that can be used as treatments for obesity or diabetes. In particular aspects, the Angptl6 peptide comprises an amino acid sequence with at least 95% identity to the amino acid sequence set forth in SEQ ID NO:1. In further aspects, the Angptl6 peptide is conjugated to a heterologous protein or peptide. For example, the heterologous protein can be selected from the group consisting of human serum albumin, immunoglobulin, Fc fragment of an immunoglobulin, and transferrin. In other aspects, the Angptl6 peptide compounds comprises a fusion protein comprising the Angptl6 peptide is fused to a heterologous protein or peptide, for example, the Fc domain of an immunoglobulin or a Flag or hexahistidine tag or a leader peptide. The fusion protein and may further contain a linker or “hinge” amino acid sequence such as the amino acids ERKCCVECPPCP (SEQ ID NO:17) or VECPPCP (SEQ ID NO:18) or GGGERKCCVECPPCP (SEQ ID NO:19) or GGGVECPPCP (SEQ ID NO:20) between the heterologous protein or peptide and the Angptl6 peptide.

In a further aspect, the present invention provides Angptl6 compounds that have the formula (I)

Z¹-peptide-Z²

wherein the peptide is the Angptl6 peptide comprising the coiled-coil domain of an Angptl6 protein and excluding an intact globular fibrinogen domain of the Angptl6 protein, wherein one or more of the amino acids can be a D- or L-amino acid, an amino acid analog, or an amino acid derivative; and Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof. In particular embodiments, the Angptl6 peptide comprises an amino acid sequence with at least 95% identity to the amino acid sequence set forth in SEQ ID NO:1. The Angptl6 peptide can further include an additional 1 to 25 amino acids between Z¹ and the peptide.

In further aspects of the above Angptl6 peptide compounds, the N-terminal amino acid of the peptide is covalently joined to one or more molecules selected from the group consisting of PEG, cholesterol, N-ethylmaleimidyl, and palmitoyl. In further still aspects of the Angptl6 peptide compounds, the peptide further includes a cysteine residue at the N-terminus of the peptide to which is optionally present a protecting group that, if present, is joined to the N-terminal amino group of the cysteine residue. In particular aspects of the peptide, the thiol group of the cysteine residue at the N-terminus is covalently joined to one or more molecules selected from the group consisting of PEG, cholesterol, N-ethylmaleimidyl, and palmitoyl. In a specific embodiment, the Angptl6 peptide compound has the amino acid of SEQ ID NO:1, which further includes a cysteine residue at the N-terminus of the peptide to which is present a protecting group joined to the N-terminal amino group of the cysteine residue and a PEG molecule joined to the thiol group.

The present invention further provides for the use of any one or more of the embodiments and aspects of the Angptl6 peptide compounds in the manufacture of a medicament for treatment of a metabolic disorder. Disorders include, but are not limited to, obesity, metabolic syndrome or syndrome X, and type II diabetes. Complications of diabetes such as retinopathy may be positively affected thereby as well. Obesity is a comorbidity of and may well contribute to such disease states as diabetes, hypertension, dyslipidemias, cardiovascular disease, gallstones, osteoarthritis, insulin resistance, and certain forms of cancers. Thus, the present invention provides a composition comprising one or more of any of the above Angptl6 peptide compounds and a pharmaceutically acceptable carrier.

### BRIEF DESCRIPTION OF THE DRAWINGS

FIG. 1 is a graph showing body weight change in mice administered either a single IV dose of adenovirus-mouse angptl6 (Ad-Angptl6) or adenovirus-GFP (Ad-GFP). The X-axis indicates days after injection and the y axis indicates body weight in grams. An * indicates a significant (p<0.05) change between the two groups.

FIG. 2 is a graph comparing body weight lost 17 days after a single IV dose of adenovirus-mouse angptl6 (Ad-Angptl6) or adenovirus-GFP (Ad-GFP). The Y axis indicates weight lost in grams. An ** indicates a significant (p<0.05) change between the two groups.

FIG. 3 is a graph of daily food intake in mice administered either a single IV dose of adenovirus-mouse angptl6 (Ad-Angptl6) or adenovirus-GFP (Ad-GFP). The X-axis indicates days after injection and the Y axis indicates food consumed in grams per day. An * indicates a significant (p<0.05) change between the two groups.

FIG. 4 is a graph of weight change in mice administered either a single IV dose of adenovirus-mouse angptl6 (Ad-Angptl6) or adenovirus-GFP (Ad-GFP). The X-axis indicates days after injection and the Y axis indicates weight loss in grams from the beginning of the study. An * indicates a significant (p<0.05) change between the two groups.

FIG. 5 is a graph of daily food intake in mice administered either a single IV dose of saline, control vector (Ad-pterm), adenovirus-mouse angptl6 (Ad-Angptl6), or adenovirus-N-terminal mouse Angtpl6 (Ad-NAngptl6). The X-axis indicates days after injection and the y axis indicates food consumed in grams per day. An * indicates a significant (p<0.05) change relative to the Ad-Pterm group.

FIG. 6 is a graph of weight change in mice administered either a single IV dose of saline, control vector (Ad-pterm), adenovirus-mouse angptl6 (Ad-Angptl6), or adenovirus-N-terminal mouse Angtpl6 (Ad-NAngptl6). The X-axis indicates days after injection and the Y axis indicates weight loss in grams from the beginning of the study. An * indicates a significant (p<0.05) change relative to the Ad-Pterm group.

FIG. 7 is a graph of the weight change in fat, muscle, or free fluid (FF) in mice administered either a single IV dose of saline, control vector (Ad-pterm), adenovirus-mouse angptl6 (Ad-Angptl6), or adenovirus-N-terminal mouse Angtpl6 (Ad-NAngptl6). The Y axis is the weight change 13 days after injection. An * indicates a significant (p<0.05) change relative to the Ad-Pterm group.

FIG. 8 is schematic showing the position of PCR primers used to detect expression of mouse angptl6 (Adv-Angptl6) or the N-terminal mouse Angtpl6 (Ad-NAngptl6).

FIG. 9 is a graph showing expression of N-terminal angtpl6 (Ad-NAngptl6) or angtpl6 (Ad-Angptl6) in mice administered either a single IV dose of saline, control vector (Ad-pterm), adenovirus-mouse angptl6 (Ad-Angptl6), or adenovirus-N-terminal mouse Angtpl6 (Ad-NAngptl6). The Y axis is expression relative to native angptl6 in the liver derived from Taqman data.

### DETAILED DESCRIPTION OF THE INVENTION

Angiopoietin-related growth factor, also known as Angptl16, was recently identified as an orphan 50 KD secreted protein, mainly from the liver, that acts a as an endocrine signal in the peripheral tissues. Evidence from three independent genetic models implicated Angptl6 as a compound for treatment of obesity and insulin resistance (Oike et al., Nat. Med. 11: 400408 (2005)). Angptl6 KO mice are severely obese, while transgenic mice overexpressing Angptl6 are resistant to diet-induced obesity and show an improvement in insulin sensitivity. Furthermore, diet-induced obese (DIO) mice treated with adenoviral vectors expressing Angptl6 exhibited weight loss and correction of diabetes. However, in addition to Angptl6's role in metabolic disorders, Angptl6 has been identified as a pro-angiogenesis agent in vitro as well as in vivo. Angptl6 like other members of the Angptl family have a characteristic structure: signal peptide, an extended domain predicted to form a dimeric or trimeric coiled-coil, and a globular fibrinogen domain. We hypothesized that, like other members of the Angptl family (Angptl3 and Angptl4), Angptl6's structural domains might posses independent functions. Adenovirus (Ad) vectors overexpressing full-length Angptl6 or N-terminus portion of the protein (containing the coiled-coil domain) were constructed and tested in vivo. Previously in WO2005097171 and Oike et al., Nat. Med. 11: 400-408 (2005) it was shown that a full-length Angptl6 protein reduced obesity and insulin resistance, which suggested Angptl6 protein could be used as an antiobesity agent. However, full-length Angptl6 protein also causes angiogenesis, an unacceptable effect for an antiobesity treatment. The inventors show herein that expression of a subdomain of Angptl6 protein comprising the coiled-coil domain and not the globular fibrinogen domain reduces obesity and insulin resistance but without the undesirable angiogenesis side effects.

Thus, the present invention provides angiopoietin-like protein 6 (Angptl6) peptide compounds comprising the coiled-coil domain and excluding an intact globular fibrinogen domain of Angptl6 and compositions thereof that can be used as treatments for metabolic disorders. One or more of the Angptl6 peptide compounds can be administered to an individual to treat a metabolic disorder afflicting the individual. Such disorders include, but are not limited to, obesity, metabolic syndrome or syndrome X, and type II diabetes. Complications of diabetes such as retinopathy may be positively affected thereby as well. Obesity is a comorbidity of and may well contribute to such disease states as diabetes, hypertension, dyslipidemias, cardiovascular disease, gallstones, osteoarthritis and certain forms of cancers. Administration of one or more of the Angptl6 peptide compounds disclosed herein to effect weight loss in an individual may also be useful in preventing such diseases and as part of therapy for any one of the above-recited conditions, as well as others. In other embodiments, there is provided a method for treating a metabolic disease in an individual comprising administering to the individual a one or more of the Angptl6 peptide compounds described above. The metabolic disease may be selected from the group consisting of diabetes, metabolic syndrome, hyperglycemia, and obesity and may be administered via a route peripheral to the brain, such as an oral, mucosal, buccal, sublingual, nasal, rectal, subcutaneous, transdermal, intravenous, intramuscular, or intraperitoneal route. In particular embodiments, the Angptl6 peptide compounds can be used to treat multiple disorders in an individual. As will be apparent to one of ordinary skill in the art in view of the disclosure herein, the Angptl6 peptide compounds can be administered to an individual to effect a reduction in food intake by the individual, to effect a reduction in weight gain in the individual, to prevent weight gain in the individual, to effect weight loss in the individual, and/or to prevent weight regain in the individual.

Accordingly, the present invention provides Angptl6 peptide compounds comprising the coiled-coil domain of an Angptl6 proteins and excluding an intact globular fibrinogen domain of the Angptl6 protein and compositions thereof that can be used as treatments for obesity or diabetes. In particular aspects, the Angptl6 peptide comprises an amino acid sequence with at least 95% identity to the amino acid sequence set forth in SEQ ID NO:1. In further aspects, the Angptl6 peptide can further include its endogenous leader peptide at the amino terminus or a heterologous peptide at the amino terminus or the carboxy terminus. In particular aspects, the heterologous peptide is a leader peptide at the amino terminus that facilitates secretion of the peptide from a cell. In further aspects, the leader sequence is joined or fused to the Angptl6 peptide by a peptide that includes a cleavage site for removing the leader peptide from Angptl6 peptide. In further aspects, the Angptl6 peptide is conjugated to a heterologous protein or peptide. For example, the heterologous protein can be selected from the group consisting of human serum albumin, immunoglobulin, and transferrin. In other aspects, the Angptl6 peptide compound comprises a fusion protein comprising the Angptl6 peptide fused at its C- or N-terminus to a heterologous protein or peptide, for example, the Fc domain or moiety of an immunoglobulin or a Flag or hexahistidine tag or a leader peptide. The Fc domain can be derived from mouse IgG₁ or human IgG₂M4. Human IgG₂M4 is an antibody from IgG₂ with mutations with which the antibody maintains normal pharmacokinetic profile but does not possess any known effector function (See U.S. Published Application No. 20070148167 and U.S. Published Application No. 20060228349). The fusion protein and may further contain a linker or “hinge” amino acid sequence such as the amino acids ERKCCVECPPCP (SEQ ID NO:17) or VECPPCP (SEQ ID NO:18) or GGGERKCCVECPPCP (SEQ ID NO:19) or GGGVECPPCP (SEQ ID NO:20) between the heterologous protein or peptide and the Angptl6 peptide. The Angptl6 peptide can be expressed in E. coli, yeast (such as Pichia pastoris or Saccharomyces cerevisiae), or mammalian cells.

In a further aspect, the present invention provides Angptl6 compounds that have the formula (I)

Z¹-peptide-Z²

wherein the peptide is the Angptl6 peptide comprising the coiled-coil domain of an Angptl6 protein and excluding an intact globular fibrinogen domain of the Angptl6 protein, wherein one or more of the amino acids can be a D- or L-amino acid, an amino acid analog, or an amino acid derivative; and Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof. In particular embodiments, the Angptl6 peptide comprises an amino acid sequence with at least 95% identity to the amino acid sequence set forth in SEQ ID NO:1. The Angptl6 peptide can further include an endogenous or heterologous leader peptide or any heterologous peptide from 1 to 25 amino acids.

In particular aspects, the Angptl6 peptide compound optionally includes a protecting group covalently joined to the N-terminal amino group of the Angptl6 peptide. A protecting group covalently joined to the N-terminal amino group of the Angptl6 peptide reduces the reactivity of the amino terminus under in vivo conditions. Amino protecting groups include —C₁₋₁₀ alkyl, —C₁₋₁₀ substituted alkyl, —C₂₋₁₀ alkenyl, —C₂₋₁₀ substituted alkenyl, aryl, —C₁₋₆ alkyl aryl, —C(O)—(CH₂)₁₋₆—COOH, —C(O)—C₁₋₆ alkyl, —C(O)-aryl, —C(O)—O—C₁₋₆ alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. Deamination of the N-terminal amino acid is another modification that is contemplated for reducing the reactivity of the amino terminus under in vivo conditions.

Chemically modified compositions of the Angptl6 peptide compounds wherein the Angptl6 peptide is linked to a polymer are also included within the scope of the present invention. The polymer selected is usually modified to have a single reactive group, such as an active ester for acylation or an aldehyde for alkylation, so that the degree of polymerization may be controlled as provided for in the present methods. Included within the scope of polymers is a mixture of polymers. Preferably, for therapeutic use of the end-product preparation, the polymer will be pharmaceutically acceptable.

The polymer or mixture thereof may be selected from the group consisting of, for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In further still embodiments, the Angptl6 peptide is modified by PEGylation, cholesteroylation, or palmitoylation. The modification can be to any amino acid residue in the Angptl6 peptide, however, in currently preferred embodiments, the modification is to the N-terminal amino acid of the Angptl6 peptide, either directly to the N-terminal amino acid or by way coupling to the thiol group of a cysteine residue added to the N-terminus or a linker added to the N-terminus such as Ttds. In further embodiments, the N-terminus of the Angptl6 peptide comprises a cysteine residue to which a protecting group is coupled to the N-terminal amino group of the cysteine residue and the cysteine thiolate group is derivatized with N-ethylmaleimide, PEG group, cholesterol group, or palmitoyl group. In further still embodiments, an acetylated cysteine residue is added to the N-terminus of the Angptl6 peptide, and the thiol group of the cysteine is derivatized with N-ethylmaleimide, PEG group, cholesterol group, or palmitoyl group.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271: 21969-21977 (1996)). Therefore, it is envisioned that the core peptide residues can be PEGylated to provide enhanced therapeutic benefits such as, for example, increased efficacy by extending half-life in vivo. Thus, PEGylating the Angptl6 peptide will improve the pharmacokinetics and pharmacodynamics of the Angtl6 peptide compound.

Peptide PEGylation methods are well known in the literature and described in the following references, each of which is incorporated herein by reference: Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., Int. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C₁₋₁₀) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG₂-NHS-40k (Nektar); mPEG₂-MAL-40k (Nektar), SUNBRIGHT GL2-400MA ((PEG)₂40 kDa) (NOF Corporation, Tokyo), SUNBRIGHT ME-200MA (PEG20kDa) (NOF Corporation, Tokyo). The PEG groups are generally attached to the Angptl6 peptides via acylation or reductive alkylation through a reactive group on the PEG moiety (for example, an aldehyde, amino, thiol, or ester group) to a reactive group on the Angptl6 peptide (for example, an aldehyde, amino, thiol, or ester group).

The PEG molecule(s) may be covalently attached to any Lys, Cys, or K(CO(CH₂)₂SH) residues at any position in the Angptl6 peptide. The Angptl6 peptide described herein can be PEGylated directly to any amino acid at the N-terminus by way of the N-terminal amino group. A “linker arm” may be added to the Angptl6 peptide to facilitate PEGylation. PEGylation at the thiol side-chain of cysteine has been widely reported (See, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. Those Angptl6 peptide, which have been PEGylated, have been PEGylated through the side chains of a cysteine residue added to the N-terminal amino acid.

Alternatively, the PEG molecule(s) may be covalently attached to an amide group in the C-terminus of the Angptl6 peptide. In general, there is at least one PEG molecule covalently attached to the Angptl6 peptide. In particular aspects, the PEG molecule is branched while in other aspects, the PEG molecule may be linear. In particular aspects, the PEG molecule is between 1 kDa and 100 kDa in molecular weight. In further aspects, the PEG molecule is selected from 10, 20, 30, 40, 50 and 60 kDa. In further still aspects, it is selected from 20, 40, or 60 kDa. Where there are two PEG molecules covalently attached to the Angptl6 peptide of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the Angptl6 peptide contains mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

Currently, it is preferable that the Angptl6 peptide is PEGylated through the side chains of a cysteine added to the N-terminal amino acid. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

A useful strategy for the PEGylation of synthetic Angptl6 peptide consists of combining, through forming a conjugate linkage in solution, a peptide, and a PEG moiety, each bearing a special functionality that is mutually reactive toward the other. The Angptl6 peptides can be easily prepared with conventional solid phase synthesis. The Angptl6 peptide is “preactivated” with an appropriate functional group at a specific site. The precursors are purified and fully characterized prior to reacting with the PEG moiety. Conjugation of the Angptl6 peptide with PEG usually takes place in aqueous phase and can be easily monitored by reverse phase analytical HPLC. The PEGylated Angptl6 peptide can be easily purified by cation exchange chromatography or preparative HPLC and characterized by analytical HPLC, amino acid analysis and laser desorption mass spectrometry.

The Angptl6 peptide compounds can comprise other non-sequence modifications, for example, glycosylation, lipidation, acetylation, phosphorylation, carboxylation, methylation, or any other manipulation or modification, such as conjugation with a labeling component. While, in particular aspects, the Angptl6 peptide compounds herein utilize naturally-occurring amino acids or D isoforms of naturally occurring amino acids, substitutions with non-naturally occurring amino acids (for example, methionine sulfoxide, methionine methylsulfonium, norleucine, epsilon-aminocaproic acid, 4-aminobutanoic acid, tetrahydroisoquinoline-3-carboxylic acid, 8-aminocaprylic acid, 4 aminobutyric acid, Lys(N(epsilon)-trifluoroacetyl) or synthetic analogs, for example, o-aminoisobutyric acid, p or y-amino acids, and cyclic analogs.

In further still aspects, the Angptl6 peptide compounds comprise a fusion protein that having a first moiety, which is a Angptl6 peptide, and a second moiety, which is a heterologous peptide or protein. Fusion proteins may include myc-, HA-, or His6-tags. Fusion proteins further include the Angptl6 peptide fused to the Fc domain of a human IgG. In particular aspects, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130. The Fc moiety can be derived from mouse IgG1 or human IgG₂M4. Human IgG₂M4 (See U.S. Published Application No. 20070148167 and U.S. Published Application No. 20060228349) is an antibody from IgG₂ with mutations with which the antibody maintains normal pharmacokinetic profile but does not possess any known effector function. Fusion proteins further include the Angptl6 peptide fused to human serum albumin, transferrin, or an antibody.

In further still aspects, the Angptl6 peptide compounds include embodiments wherein the Angtl6 peptide is conjugated to a carrier protein such as human serum albumin, transferring, or an antibody molecule.

The Angptl6 peptide compounds may be modified by a variety of chemical techniques to produce derivatives having essentially the same activity as the unmodified Angptl6 protein or peptide and/or having other desirable properties. A protecting group covalently joined to the C-terminal carboxy group reduces the reactivity of the carboxy terminus under in vivo conditions. For example, carboxylic acid groups of the peptide, whether carboxyl-terminal or side chain, may be provided in the form of a salt of a pharmacologically-acceptable cation or esterified to form a C1-6 ester, or converted to an amide of formula NRR2 wherein R and R2 are each independently H or C1-6 alkyl, or combined to form a heterocyclic ring, such as a 5- or 6-membered ring. The carboxy terminus protecting group is preferably attached to the α-carbonyl group of the last amino acid. Carboxy terminus protecting groups include, but are not limited to, amide, methylamide, and ethylamide. Amino groups of the peptide, whether N-terminal or side chain, may be in the form of a pharmacologically-acceptable acid addition salt, such as the HCl, HBr, acetic, benzoic, toluene sulfonic, maleic, tartaric, and other organic salts, or may be modified to C1-6 alkyl or dialkyl amino or further converted to an amide.

Hydroxyl groups of the Angptl6 peptide side chain may be converted to C1-6 alkoxy or to a C1-6 ester using well-recognized techniques. Phenyl and phenolic rings of the peptide side chain may be substituted with one or more halogen atoms, such as fluorine, chlorine, bromine or iodine, or with C1-6 alkyl, C1-6 alltoxy, carboxylic acids and esters thereof, or amides of such carboxylic acids. Methylene groups of the Angptl6 peptide side chains can be extended to homologous C2-4 alkylenes. Thiols can be protected with any one of a number of well-recognized protecting groups, such as acetamide groups. Those skilled in the art will also recognize methods for introducing cyclic structures into the Angptl6 peptide to select and provide conformational constraints to the structure that result in enhanced stability. For example, a carboxyl-terminal or amino-terminal cysteine residue can be added to the Angptl6 peptide, so that when oxidized, the Angptl6 peptide will contain a disulfide bond, thereby generating a cyclic peptide. Other peptide cyclizing methods include the formation of thioethers and carboxyl- and amino-terminal amides and esters.

Polysaccharide polymers are another type of water soluble polymer that may be used for protein modification. Dextrans are polysaccharide polymers comprised of individual subunits of glucose predominantly linked by α1-6 linkages. The dextran itself is available in many molecular weight ranges, and is readily available in molecular weights from about 1 kDa to about 70 kDa. Dextran is a suitable water soluble polymer for use as a vehicle by itself or in combination with another vehicle (See, for example, WO 96/11953 and WO 96/05309). The use of dextran conjugated to therapeutic or diagnostic immunoglobulins has been reported; see, for example, European Patent Publication No. 0 315 456. Dextran of about 1 kDa to about 20 kDa is preferred when dextran is used as a vehicle in accordance with the present invention.

As described above, the presence of a “linker” group is optional. When present, its chemical structure is not critical, since it serves primarily as a spacer. However, in certain embodiments, the linker may itself provide improved properties to the compositions of the present invention. The linker is preferably made up of amino acids linked together by peptide bonds. Thus, in particular embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines. Other specific examples of linkers are (Gly)₃(Gly)₄; (Gly)₃AsnGlySer(Gly)₂; (Gly)₃Cys(Gly)₄; and GlyProAsnGlyGly.

Non-peptide linkers can also be used. For example, alkyl linkers such as —NH—(CH₂)ₛ—C(O)—, wherein s=2-20 could be used. These alkyl linkers may further be substituted by any non-sterically hindering group such as lower alkyl (for example, C₁₋₆) lower acyl, halogen (for example, Cl, Br), CN, NH₂, phenyl, and the like. An exemplary non-peptide linker is a PEG linker, wherein n is such that the linker has a molecular weight of 100 to 5000 kD, preferably 100 to 500 kD. The peptide linkers may be altered to form derivatives in the same manner as described above. Other linkers include Ttds (1-amino-4,7,10-trioxa-13-tridecanamine succinimic acid).

The present invention includes diastereomers as well as their racemic and resolved enantiomerically pure forms. The Angptl6 peptides can contain D-amino acids, L-amino acids, or a combination thereof. In general, the amino acids are in the L-form with particular amino acids in D-form. As is known in the art, individual amino acids can be represented as follows: A=Ala=Alanine; C=Cys=Cysteine; D=Asp=Aspartic Acid; E=Glu=Glutamic Acid; F=Phe=Phenylalanine; G=Gly=Glycine; H=His=Histidine; I=Ile=Isoleucine; K=Lys=Lysine; L=Leu=Leucine; M=Met=Methionine; N=Asn=Asparagine; P=Pro=Proline; Q=Gln=Glutamine; R=Arg=Arginine; S=Ser=Serine; T=Thr=Threonine; V=Val=Valine; W=Trp=Tryptophan; and Y=Tyr=Tyrosine.

Further provided are pharmaceutical compositions comprising a therapeutically effective amount of one or more of the Angptl6 peptide compounds disclosed herein for the treatment of a metabolic disorder in an individual. Such disorders include, but are not limited to, obesity, metabolic syndrome or syndrome X, type II diabetes, complications of diabetes such as retinopathy, hypertension, dyslipidemias, cardiovascular disease, gallstones, osteoarthritis, insulin resistance, and certain forms of cancers. The obesity-related disorders herein are associated with, caused by, or result from obesity.

“Obesity” is a condition in which there is an excess of body fat. The operational definition of obesity is based on the Body Mass Index (BMI), calculated as body weight per height in meters squared (kg/m2). “Obesity” refers to a condition whereby an otherwise healthy subject has a Body Mass Index (BMI) greater than or equal to 30 kg/m2, or a condition whereby a subject with at least one co-morbidity has a BMI greater than or equal to 27 kg/m2. An “obese subject” is an otherwise healthy subject with a Body Mass Index (BMI) greater than or equal to 30 kg/m2 or a subject with at least one co-morbidity with a BMI greater than or equal to 27 kg/m2. A “subject at risk for obesity” is an otherwise healthy subject with a BMI of 25 kg/m2 to less than 30 kg/m2 or a subject with at least one co-morbidity with a BMI of 25 kg/m2 to less than 27 kg/m2.

The increased risks associated with obesity occur at a lower Body Mass Index (BMI) in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m2. In Asian countries, including Japan, an “obese subject” refers to a subject with at least one obesity-induced or obesity-related co-morbidity that requires weight reduction or that would be improved by weight reduction, with a BMI greater than or equal to 25 kg/m2. In Asian countries, a “subject at risk of obesity” is a subject with a BMI of greater than 23 kg/m2 to less than 25 kg/m2.

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The term “diabetes,” as used herein, includes both insulin-dependent diabetes mellitus (IDDM, also known as type I diabetes) and non-insulin-dependent diabetes mellitus (NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compounds of the present invention are useful for treating both Type I and Type II diabetes. The compounds are especially effective for treating Type II diabetes. The compounds of the present invention are also useful for treating and/or preventing gestational diabetes mellitus.

The Angptl6 peptide compounds disclosed herein may be used in a pharmaceutical composition when combined with a pharmaceutically acceptable carrier. Such compositions comprise a therapeutically-effective amount of the Angptl6 peptide compound and a pharmaceutically acceptable carrier. Such a composition may also be comprised of (in addition to Angptl6 peptide compound and a carrier) diluents, fillers, salts, buffers, stabilizers, solubilizers, and other materials well known in the art. Compositions comprising the Angptl6 peptide compound can be administered, if desired, in the form of salts provided the salts are pharmaceutically acceptable. Salts may be prepared using standard procedures known to those skilled in the art of synthetic organic chemistry.

The term “individual” is meant to include humans and companion or domesticated animals such as dogs, cats, horses, and the like. Therefore, the compositions comprising formula I are also useful for treating or preventing obesity and obesity-related disorders in cats and dogs. As such, the term “mammal” includes companion animals such as cats and dogs.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to the Angptl6 peptide compounds of the general formula (I) are meant to also include the pharmaceutically acceptable salts.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration. The Angptl6 peptide compounds may include multimers (for example, heterodimers or homodimers) or complexes with itself or other peptides. As a result, pharmaceutical compositions of the invention may comprise one or more Angptl6 peptide compounds in such multimeric or complexed form.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially, or simultaneously.

The pharmacological composition can comprise one or more Angptl6 peptide compounds; one or more Angptl6 peptide compounds and one or more other agents for treating a metabolic disorder; or the pharmacological composition comprising the one or more Angptl6 peptide compounds can be used concurrently with a pharmacological composition comprising an agent for treating a metabolic disorder. Such disorders include, but are not limited to, obesity, metabolic syndrome or syndrome X, type II diabetes, complications of diabetes, hypertension, dyslipidemias, cardiovascular disease, gallstones, osteoarthritis, insulin resistance, and certain forms of cancers.

When the pharmacological composition comprises another agent for treating a metabolic disorder or the treatment includes a second pharmacological composition comprising an agent for treating a metabolic disorder, the agent includes, but are not limited to, other injectable products for obesity and diabetes, such as peptides, antibodies, and proteins. Agents that improve metabolic disorders, such as Adiponectin, as well as antibodies that cause weight loss or improved glycemic control (such as a ghrelin antibody, myostatin antibody, anti-PCI, anti-Fetuin, etc) are contemplated. Further contemplated are agents such as cannabinoid (CB1) receptor antagonists, glucagon like peptide 1 (GLP-1) receptor agonists, Byetta, Oxyntomodulin derivatives, NMU derivatives and analogs, NMS derivatives and analogs, leptin, PYY3-36 derivatives, PP derivatives, amylin derivatives lipase inhibitors, tetrahydrolipstatin, 2-4-dinitrophenol, acarbose, sibutramine, phentamine, fat absorption blockers, simvastatin, mevastatin, ezetimibe, atorvastatin, sitagliptin, metformin, orlistat, Qnexa, topiramate, naltrexone, bupriopion, phentermine, losartan, losartan with hydrochlorothiazide, and the like.

Suitable agents of use in combination with the Angptl6 peptide compounds, include, but are not limited to:

(a) anti-diabetic agents such as (1) PPARγ agonists such as glitazones (e.g. ciglitazone; darglitazone; englitazone; isaglitazone (MCC-555); pioglitazone (ACTOS); rosiglitazone (AVANDIA); troglitazone; rivoglitazone, BRL49653; CLX-0921; 5-BTZD, GW-0207, LG-100641, R483, and LY-300512, and the like and compounds disclosed in WO97/10813, 97/27857, 97/28115, 97/28137, 97/27847, 03/000685, and 03/027112 and SPPARMS (selective PPAR gamma modulators) such as T131 (Amgen), FK614 (Fujisawa), netoglitazone, and metaglidasen; (2) biguanides such as buformin; metformin; and phenformin, and the like; (3) protein tyrosine phosphatase-1B (PTP-1B) inhibitors such as ISIS 113715, A-401674, A-364504, IDD-3, IDD 2846, KP-40046, KR61639, MC52445, MC52453, C7, OC-060062, OC-86839, OC29796, TTP-277BC1, and those agents disclosed in WO 04/041799, 04/050646, 02/26707, 02/26743, 04/092146, 03/048140, 04/089918, 03/002569, 04/065387, 04/127570, and US 2004/167183; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, metiglinide (GLUFAST) and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and Al-3688, and the like; (8) insulin secreatagogues such as linogliride nateglinide, mitiglinide (GLUFAST), ID1101 A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics, such as biota, LP-100, novarapid, insulin detemir, insulin lispro, insulin glargine, insulin zinc suspension (lente and ultralente); Lys-Pro insulin, GLP-1 (17-36), GLP-1 (73-7) (insulintropin); GLP-1 (7-36)-NH2) exenatide/Exendin-4, Exenatide LAR, Linaglutide, AVE0010, CJC 1131, BIM51077, CS 872, THO318, BAY-694326, GP010, ALBUGON (GLP-1 fused to albumin), HGX-007 (Epac agonist), S-23521, and compounds disclosed in WO 04/022004, WO 04/37859, and the like; (12) non-thiazolidinediones such as JT-501, and farglitazar (GW-2570/GI-262579), and the like; (13) PPARα/γ dual agonists such as AVE 0847, CLX-0940, GW-1536, GW1929, GW-2433, KRP-297, L-796449, LBM 642, LR-90, LY510919, MK-0767, ONO 5129, SB 219994, TAK-559, TAK-654, 677954 (GlaxoSmithkline), E-3030 (Eisai), LY510929 (Lilly), AK109 (Asahi), DRF2655 (Dr. Reddy), DRF8351 (Dr. Reddy), MC3002 (Maxocore), TY51501 (ToaEiyo), naveglitazar, muraglitizar, peliglitazar, tesaglitazar (GALIDA), reglitazar (JTT-501), chiglitazar, and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453; and (14) other insulin sensitizing drugs; (15) VPAC2 receptor agonists; (16) GLK modulators, such as PSN105, RO 281675, RO 274375 and those disclosed in WO 03/015774, WO 03/000262, WO 03/055482, WO 04/046139, WO 04/045614, WO 04/063179, WO 04/063194, WO 04/050645, and the like; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine, CT21022, CT20026, CT-98023, SB-216763, SB410111, SB-675236, CP-70949, XD4241 and those compounds disclosed in WO 03/037869, 03/03877, 03/037891, 03/024447, 05/000192, 05/019218 and the like; (19) glycogen phosphorylase (HGLPa) inhibitors, such as AVE 5688, PSN 357, GPi-879, those disclosed in WO 03/037864, WO 03/091213, WO 04/092158, WO 05/013975, WO 05/013981, US 2004/0220229, and JP 2004-196702, and the like; (20) ATP consumption promoters such as those disclosed in WO 03/007990; (21) fixed combinations of PPARγ agonists and metformin such as AVANDAMET; (22) PPAR pan agonists such as GSK 677954; (23) GPR40 (G-protein coupled receptor 40) also called SNORF 55 such as BG 700, and those disclosed in WO 04/041266, 04/022551, 03/099793; (24) GPR119 (also called RUP3; SNORF 25) such as RUP3, HGPRBMY26, PFI 007, SNORF 25; (25) adenosine receptor 2B antagonists such as ATL-618, ATl-802, E3080, and the like; (26) carnitine palmitoyl transferase inhibitors such as ST 1327, and ST 1326, and the like; (27) Fructose 1,6-bisphosphohatase inhibitors such as CS-917, MB7803, and the like; (28) glucagon antagonists such as AT77077, BAY 694326, GW 4123X, NN2501, and those disclosed in WO 03/064404, WO 05/00781, US 2004/0209928, US 2004/029943, and the like; (30) glucose-6-phosphase inhibitors; (31) phosphoenolpyruvate carboxykinase (PEPCK) inhibitors; (32) pyruvate dehydrogenase kinase (PDK) activators; (33) RXR agonists such as MC1036, CS00018, JNJ 10166806, and those disclosed in WO 04/089916, U.S. Pat. No. 6,759,546, and the like; (34) SGLT inhibitors such as AVE 2268, KGT 1251, T1095/RWJ 394718; (35) BLX-1002;

(b) lipid lowering agents such as (1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, itavastatin, pitavastatin, fluvastatin, lovastatin, pravastatin, rivastatin, rosuvastatin, simvastatin, rosuvastatin (ZD-4522), and the like, particularly simvastatin; (3) HMG-CoA synthase inhibitors; (4) cholesterol absorption inhibitors such as FMVP4 (Forbes Medi-Tech), KT6-971 (Kotobuki Pharmaceutical), FM-VA12 (Forbes Medi-Tech), FM-VP-24 (Forbes Medi-Tech), stanol esters, beta-sitosterol, sterol glycosides such as tiqueside; and azetidinones such as ezetimibe, and those disclosed in WO 04/005247 and the like; (5) acyl coenzyme A-cholesterol acyl transferase (ACAT) inhibitors such as avasimibe, eflucimibe, pactimibe (KY505), SMP 797 (Sumitomo), SM32504 (Sumitomo), and those disclosed in WO 03/091216, and the like; (6) CETP inhibitors such as JTT 705 (Japan Tobacco), torcetrapib, CP 532,632, BAY63-2149 (Bayer), SC 591, SC 795, and the like; (7) squalene synthetase inhibitors; (8) anti-oxidants such as probucol, and the like; (9) PPARα agonists such as beclofibrate, benzafibrate, ciprofibrate, clofibrate, etofibrate, fenofibrate, gemcabene, and gemfibrozil, GW 7647, BM 170744 (Kowa), LY518674 (Lilly), GW590735 (GlaxoSmithkline), KRP-101 (Kyorin), DRF10945 (Dr. Reddy), NS-220/R1593 (Nippon Shinyaku/Roche, ST1929 (Sigma Tau) MC3001/MC3004 (MaxoCore Pharmaceuticals, gemcabene calcium, other fibric acid derivatives, such as Atromid®, Lopid®, and Tricor®, and those disclosed in U.S. Pat. No. 6,548,538, and the like; (10) FXR receptor modulators such as GW 4064 (GlaxoSmithkline), SR 103912, QRX401, LN-6691 (Lion Bioscience), and those disclosed in WO 02/064125, WO 04/045511, and the like; (11) LXR receptor modulators such as GW 3965 (GlaxoSmithkline), T9013137, and XTCO179628 (X-Ceptor Therapeutics/Sanyo), and those disclosed in WO 03/031408, WO 03/063796, WO 04/072041, and the like; (12) lipoprotein synthesis inhibitors such as niacin; (13) renin angiotensin system inhibitors; (14) PPAR δ partial agonists, such as those disclosed in WO 03/024395; (15) bile acid reabsorption inhibitors, such as BARI 1453, SC435, PHA384640, S8921, AZD7706, and the like; and bile acid sequesterants such as colesevelam (WELCHOL/CHOLESTAGEL), (16) PPARγ agonists such as GW 501516 (Ligand, GSK), GW 590735, GW-0742 (GlaxoSmithkline), T659 (Amgen/Tularik), LY934 (Lilly), NNC610050 (Novo Nordisk) and those disclosed in WO97/28149, WO 01/79197, WO 02/14291, WO 02/46154, WO 02/46176, WO 02/076957, WO 03/016291, WO 03/033493, WO 03/035603, WO 03/072100, WO 03/097607, WO 04/005253, WO 04/007439, and JP10237049, and the like; (17) triglyceride synthesis inhibitors; (18) microsomal triglyceride transport (MTTP) inhibitors, such as implitapide, LAB687, JTT130 (Japan Tobacco), CP346086, and those disclosed in WO 03/072532, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists including HM74A receptor agonists; (25) PPAR modulators such as those disclosed in WO 01/25181, WO 01/79150, WO 02/79162, WO 02/081428, WO 03/016265, WO 03/033453; (26) niacin-bound chromium, as disclosed in WO 03/039535; (27) substituted acid derivatives disclosed in WO 03/040114; (28) infused HDL such as LUV/ETC-588 (Pfizer), APO-A1 Milano/ETC216 (Pfizer), ETC-642 (Pfizer), ISIS301012, D4F (Bruin Pharma), synthetic trimeric ApoA1, Bioral Apo A1 targeted to foam cells, and the like; (29) IBAT inhibitors such as BARI143/HMR145A/HMR1453 (Sanofi-Aventis, PHA384640E (Pfizer), S8921 (Shionogi) AZD7806 (AstrZeneca), AK105 (Asah Kasei), and the like; (30) Lp-PLA2 inhibitors such as SB480848 (GlaxoSmithkline), 659032 (GlaxoSmithkline), 677116 (GlaxoSmithkline), and the like; (31) other agents which affect lipic composition including ETC1001/ESP31015 (Pfizer), ESP-55016 (Pfizer), AGI1067 (AtheroGenics), AC3056 (Amylin), AZD4619 (AstrZeneca); and

(c) anti-hypertensive agents such as (1) diuretics, such as thiazides, including chlorthalidone, chlorothiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, bamidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as candesartan, eprosartan, irbesartan, losartan, pratosartan, tasosartan, telmisartan, valsartan, and EXP-3137, F16828K, and RNH6270, and the like; (9) α/β adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (11) alpha 2 agonists such as lofexidine, tiamenidine, moxonidine, rilmenidine and guanobenz, and the like; (12) aldosterone inhibitors, and the like; (13) angiopoietin-2-binding agents such as those disclosed in WO 03/030833; and

(d) anti-obesity agents, such as (1) 5HT (serotonin) transporter inhibitors, such as paroxetine, fluoxetine, fenfluramine, fluvoxamine, sertraline, and imipramine, and those disclosed in WO 03/00663, as well as serotonin/noradrenaline re uptake inhibitors such as sibutramine (MERIDIA/REDUCTIL) and dopamine uptake inhibitor/Norepenephrine uptake inhibitors such as radafaxine hydrochloride, 353162 (GlaxoSmithkline), and the like; (2) NE (norepinephrine) transporter inhibitors, such as GW 320659, despiramine, talsupram, and nomifensine; (3) CB1 (cannabinoid-1 receptor) antagonist/inverse agonists, such as rimonabant (ACCOMPLIA Sanofi Synthelabo), SR-147778 (Sanofi Synthelabo), AVE1625 (Sanofi-Aventis), BAY 65-2520 (Bayer), SLV 319 (Solvay), SLV326 (Solvay), CP945598 (Pfizer), E-6776 (Esteve), 01691 (Organix), ORG14481 (Organon), VER24343 (Vernalis), NESS0327 (Univ of Sassari/Univ of Cagliari), and those disclosed in U.S. Pat. Nos. 4,973,587, 5,013,837, 5,081,122, 5,112,820, 5,292,736, 5,532,237, 5,624,941, 6,028,084, and 6,509367; and WO 96/33159, WO97/29079, WO98/31227, WO 98/33765, WO98/37061, WO98/41519, WO98/43635, WO98/43636, WO99/02499, WO00/10967, WO00/10968, WO 01/09120, WO 01/58869, WO 01/64632, WO 01/64633, WO 01/64634, WO 01/70700, WO 01/96330, WO 02/076949, WO 03/006007, WO 03/007887, WO 03/020217, WO 03/026647, WO 03/026648, WO 03/027069, WO 03/027076, WO 03/027114, WO 03/037332, WO 03/040107, WO 04/096763, WO 04/111039, WO 04/111033, WO 04/111034, WO 04/111038, WO 04/013120, WO 05/000301, WO 05/016286, WO 05/066126 and EP-658546 and the like; (4) ghrelin agonists/antagonists, such as BVT81-97 (BioVitrum), RC1291 (Rejuvenon), SRD-04677 (Sumitomo), unacylated ghrelin (TheraTechnologies), and those disclosed in WO 01/87335, WO 02/08250, WO 05/012331, and the like; (5) H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO 03/024928 and WO 03/024929; (6) melanin-concentrating hormone 1 receptor (MCH1R) antagonists, such as T-226296 (Takeda), T71 (Takeda/Amgen), AMGN-608450, AMGN-503796 (Amgen), 856464 (GlaxoSmithkline), A224940 (Abbott), A798 (Abbott), ATC0175/AR224349 (Arena Pharmaceuticals), GW803430 (GlaxoSmithkline), NBI-1A (Neurocrine Biosciences), NGX-1 (Neurogen), SNP-7941 (Synaptic), SNAP9847 (Synaptic), T-226293 (Schering Plough), TPI-1361-17 (Saitama Medical School/University of California Irvine), and those disclosed WO 01/21169, WO 01/82925, WO 01/87834, WO 02/051809, WO 02/06245, WO 02/076929, WO 02/076947, WO 02/04433, WO 02/51809, WO 02/083134, WO 02/094799, WO 03/004027, WO 03/13574, WO 03/15769, WO 03/028641, WO 03/035624, WO 03/033476, WO 03/033480, WO 04/004611, WO 04/004726, WO 04/011438, WO 04/028459, WO 04/034702, WO 04/039764, WO 04/052848, WO 04/087680; and Japanese Patent Application Nos. JP 13226269, JP 1437059, JP2004315511, and the like; (7) MCH2R (melanin concentrating hormone 2R) agonist/antagonists; (8) NPY1 (neuropeptide Y Y1) antagonists, such as BMS205749, BIBP3226, J-115814, BIBO 3304, LY-357897, CP-671906, and GI-264879A; and those disclosed in U.S. Pat. No. 6,001,836; and WO 96/14307, WO 01/23387, WO 99/51600, WO 01/85690, WO 01/85098, WO 01/85173, and WO 01/89528; (9) NPY5 (neuropeptide Y Y5) antagonists, such as 152,804, S2367 (Shionogi), E-6999 (Esteve), GW-569180A, GW-594884A (GlaxoSmithkline), GW-587081X, GW-548118X; FR 235,208; FR226928, FR 240662, FR252384; 1229U91, GI-264879A, CGP71683A, C-75 (Fasgen) LY-377897, LY366377, PD-160170, SR-120562A, SR-120819A, S2367 (Shionogi), JCF-104, and H409/22; and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683; and EP-01010691, EP-01044970, and FR252384; and PCT Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, WO 02/051806, WO 02/094789, WO 03/009845, WO 03/014083, WO 03/022849, WO 03/028726, WO 05/014592, WO 05/01493; and Norman et al., J. Med. Chem. 43:4288-4312 (2000); (10) leptin, such as recombinant human leptin (PEG-OB, Hoffman La Roche) and recombinant methionyl human leptin (Amgen); (11) leptin derivatives, such as those disclosed in U.S. Pat. Nos. 5,552,524; 5,552,523; 5,552,522; 5,521,283; and WO 96/23513; WO 96/23514; WO 96/23515; WO 96/23516; WO 96/23517; WO 96/23518; WO 96/23519; and WO 96/23520; (12) opioid antagonists, such as nalmefene (Revex®), 3-methoxynaltrexone, naloxone, and naltrexone; and those disclosed in WO 00/21509; (13) orexin antagonists, such as SB-334867-A (GlaxoSmithkline); and those disclosed in WO 01/96302, 01/68609, 02/44172, 02/51232, 02/51838, 02/089800, 02/090355, 03/023561, 03/032991, 03/037847, 04/004733, 04/026866, 04/041791, 04/085403, and the like; (14) BRS3 (bombesin receptor subtype 3) agonists; (15) CCK-A (cholecystokinin-A) agonists, such as AR-R 15849, GI 181771, JMV-180, A-71378, A-71623, PD170292, PD 149164, SR146131, SR125180, butabindide, and those disclosed in U.S. Pat. No. 5,739,106; (16) CNTF (ciliary neurotrophic factors), such as GI-181771 (Glaxo-SmithKline); SR146131 (Sanofi Synthelabo); butabindide; and PD170,292, PD 149164 (Pfizer); (17) CNTF derivatives, such as axokine (Regeneron); and those disclosed in WO 94/09134, WO 98/22128, and WO 99/43813; (18) GHS (growth hormone secretagogue receptor) agonists, such as NN703, hexarelin, MK-0677, SM-130686, CP-424,391, L-692,429 and L-163,255, and those disclosed in U.S. Pat. No. 6,358,951, U.S. Patent Application Nos. 2002/049196 and 2002/022637; and WO 01/56592, and WO 02/32888; (19) 5HT2c (serotonin receptor 2c) agonists, such as APD3546/AR10A (Arena Pharmaceuticals), ATH88651 (Athersys), ATH88740 (Athersys), BVT933 (Biovitrum/GSK), DPCA37215 (BMS), IK264; LY448100 (Lilly), PNU 22394; WAY 470 (Wyeth), WAY629 (Wyeth), WAY161503 (Biovitrum), R-1065, VR1065 (Vernalis/Roche) YM 348; and those disclosed in U.S. Pat. No. 3,914,250; and PCT Publications 01/66548, 02/36596, 02/48124, 02/10169, 02/44152; 02/51844, 02/40456, 02/40457, 03/057698, 05/000849, and the like; (20) Mc3r (melanocortin 3 receptor) agonists; (21) Mc4r (melanocortin 4 receptor) agonists, such as CHIR86036 (Chiron), CHIR915 (Chiron); ME-10142 (Melacure), ME-10145 (Melacure), HS-131 (Melacure), NBI72432 (Neurocrine Biosciences), NNC 70-619 (Novo Nordisk), TTP2435 (Transtech) and those disclosed in PCT Publications WO 99/64002, 00/74679, 01/991752, 01/0125192, 01/52880, 01/74844, 01/70708, 01/70337, 01/91752, 01/010842, 02/059095, 02/059107, 02/059108, 02/059117, 02/062766, 02/069095, 02/12166, 02/11715, 02/12178, 02/15909, 02/38544, 02/068387, 02/068388, 02/067869, 02/081430, 03/06604, 03/007949, 03/009847, 03/009850, 03/013509, 03/031410, 03/094918, 04/028453, 04/048345, 04/050610, 04/075823, 04/083208, 04/089951, 05/000339, and EP 1460069, and US 2005049269, and JP2005042839, and the like; (22) monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO 01/27068, and WO 01/62341; (23) serotonin reuptake inhibitors, such as dexfenfluramine, fluoxetine, and those in U.S. Pat. No. 6,365,633, and WO 01/27060, and WO 01/162341; (24) GLP-1 (glucagon-like peptide 1) agonists; (25) Topiramate (Topimax®); (26) phytopharm compound 57 (CP 644,673); (27) ACC2 (acetyl-CoA carboxylase-2) inhibitors; (28) β3 (beta adrenergic receptor 3) agonists, such as rafebergron/AD9677/TAK677 (Dainippon/Takeda), CL-316,243, SB 418790, BRL-37344, L-796568, BMS-196085, BRL-35135A, CGP12177A, BTA-243, GRC1087 (Glenmark Pharmaceuticals) GW 427353 (solabegron hydrochloride), Trecadrine, Zeneca D7114, N-5984 (Nisshin Kyorin), LY-377604 (Lilly), KT07924 (Kissei), SR 59119A, and those disclosed in U.S. Pat. No. 5,705,515, U.S. Pat. No. 5,451,677; and WO94/18161, WO95/29159, WO97/46556, WO98/04526 WO98/32753, WO 01/74782, WO 02/32897, WO 03/014113, WO 03/016276, WO 03/016307, WO 03/024948, WO 03/024953, WO 03/037881, WO 04/108674, and the like; (29) DGAT1 (diacylglycerol acyltransferase 1) inhibitors; (30) DGAT2 (diacylglycerol acyltransferase 2) inhibitors; (31) FAS (fatty acid synthase) inhibitors, such as Cerulenin and C75; (32) PDE (phosphodiesterase) inhibitors, such as theophylline, pentoxifylline, zaprinast, sildenafil, aminone, milrinone, cilostamide, rolipram, and cilomilast, as well as those described in WO 03/037432, WO 03/037899; (33) thyroid hormone β agonists, such as KB-2611 (KaroBioBMS), and those disclosed in WO 02/15845; and Japanese Patent Application No. JP 2000256190; (34) UCP-1 (uncoupling protein 1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; and those disclosed in WO 99/00123; (35) acyl-estrogens, such as oleoyl-estrone, disclosed in del Mar-Grasa, M. et al., Obesity Research, 9:202-9 (2001); (36) glucocorticoid receptor antagonists, such as CP472555 (Pfizer), KB 3305, and those disclosed in WO 04/000869, WO 04/075864, and the like; (37) 11β HSD-1 (11-beta hydroxy steroid dehydrogenase type 1) inhibitors, such as BVT 3498 (AMG 331), BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO 01/90091, 01/90090, 01/90092, 02/072084, 04/011410, 04/033427, 04/041264, 04/027047, 04/056744, 04/065351, 04/089415, 04/037251, and the like; (38) SCD-1 (stearoyl-CoA desaturase-1) inhibitors; (39) dipeptidyl peptidase IV (DPP-4) inhibitors, such as isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, SK-0403; and the compounds disclosed in WO 02/083128, WO 02/062764, WO 02/14271, WO 03/000180, WO 03/000181, WO 03/000250, WO 03/002530, WO 03/002531, WO 03/002553, WO 03/002593, WO 03/004498, WO 03/004496, WO 03/005766, WO 03/017936, WO 03/024942, WO 03/024965, WO 03/033524, WO 03/055881, WO 03/057144, WO 03/037327, WO 04/041795, WO 04/071454, WO 04/0214870, WO 04/041273, WO 04/041820, WO 04/050658, WO 04/046106, WO 04/067509, WO 04/048532, WO 04/099185, WO 04/108730, WO 05/009956, WO 04/09806, WO 05/023762, US 2005/043292, and EP 1 258 476; (40) lipase inhibitors, such as tetrahydrolipstatin (orlistat/XENICAL), ATL962 (Alizyme/Takeda), GT389255 (Genzyme/Peptimmune) Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in WO 01/77094, WO 04/111004, and U.S. Pat. Nos. 4,598,089, 4,452,813, 5,512,565, 5,391,571, 5,602,151, 4,405,644, 4,189,438, and 4,242,453, and the like; (41) fatty acid transporter inhibitors; (42) dicarboxylate transporter inhibitors; (43) glucose transporter inhibitors; and (44) phosphate transporter inhibitors; (45) anorectic bicyclic compounds such as 1426 (Aventis) and 1954 (Aventis), and the compounds disclosed in WO 00/18749, WO 01/32638, WO 01/62746, WO 01/62747, and WO 03/015769; (46) peptide YY and PYY agonists such as PYY336 (Nastech/Merck), AC162352 (IC Innovations/Curis/Amylin), TM30335/TM30338 (7™ Pharma), PYY336 (Emisphere Technologies), PEGylated peptide YY3-36, those disclosed in WO 03/026591, 04/089279, and the like; (47) lipid metabolism modulators such as maslinic acid, erythrodiol, ursolic acid uvaol, betulinic acid, betulin, and the like and compounds disclosed in WO 03/011267; (48) transcription factor modulators such as those disclosed in WO 03/026576; (49) Mc5r (melanocortin 5 receptor) modulators, such as those disclosed in WO 97/19952, WO 00/15826, WO 00/15790, US 20030092041, and the like; (50) Brain derived neutotropic factor (BDNF), (51) Mc1r (melanocortin 1 receptor modulators such as LK-184 (Proctor & Gamble), and the like; (52) 5HT6 antagonists such as BVT74316 (BioVitrum), BVT5182c (BioVitrum), E-6795 (Esteve), E-6814 (Esteve), SB399885 (GlaxoSmithkline), SB271046 (GlaxoSmithkline), RO-046790 (Roche), and the like; (53) fatty acid transport protein 4 (FATP4); (54) acetyl-CoA carboxylase (ACC) inhibitors such as CP640186, CP610431, CP640188 (Pfizer); (55) C-terminal growth hormone fragments such as AOD9604 (Monash Univ/Metabolic Pharmaceuticals), and the like; (56) oxyntomodulin; (57) neuropeptide FF receptor antagonists such as those disclosed in WO 04/083218, and the like; (58) amylin agonists such as Symlin/pramlintide/AC137 (Amylin); (59) Hoodia and trichocaulon extracts; (60) BVT74713 and other gut lipid appetite suppressants; (61) dopamine agonists such as bupropion (WELLBUTRIN/GlaxoSmithkline); (62) zonisamide (ZONEGRAN/Dainippon/Elan), and the like.

Specific compounds that can be used in combination with the Angptl6 peptide compounds include specific CB1 antagonists/inverse agonists include those described in WO03/077847, including: N-[3-(4-chlorophenyl)-2(S)-phenyl-1(S)-methylpropyl]-2-(4-trifluoromethyl-2-pyrimidyloxy)-2-methylpropanamide, N-[3-(4-chlorophenyl)-2-(3-cyanophenyl)-1-methylpropyl]-2-(5-trifluoromethyl-2-pyridyloxy)-2-methylpropanamide, N-[3-(4-chlorophenyl)-2-(5-chloro-3-pyridyl)-1-methylpropyl]-2-(5-trifluoromethyl-2-pyridyloxy)-2-methylpropanamide, and pharmaceutically acceptable salts thereof; as well as those in WO05/000809, which includes the following: 3-{1-[bis(4-chlorophenyl)methyl]azetidin-3-ylidene}-3-(3,5-difluorophenyl)-2,2-dimethylpropanenitrile, 1-{1-[1-(4-chlorophenyl)pentyl]azetidin-3-yl}-1-(3,5-difluorophenyl)-2-methylpropan-2-ol. 3-((S)-(4-chlorophenyl) {3-[(1S)-1-(3,5-difluorophenyl)-2-hydroxy-2-methylpropyl]azetidin-1-yl}methyl)benzonitrile, 3-((S)-(4-chlorophenyl) {3-[(1S)-1-(3,5-difluorophenyl)-2-fluoro-2-methylpropyl]azetidin-1-yl}methyl)benzonitrile, 3-((4-chlorophenyl) {3-[1-(3,5-difluorophenyl)-2,2-dimethylpropyl]azetidin-1-yl}methyl)benzonitrile, 3-((1S)-1-{1-[(S)-(3-cyanophenyl)(4-cyanophenyl)methyl]azetidin-3-yl}-2-fluoro-2-methylpropyl)-5-fluorobenzonitrile, 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile, and 5-((4-chlorophenyl){3-[(1S)-1-(3,5-difluorophenyl)-2-fluoro-2-methylpropyl]azetidin-1-yl}methyl)thiophene-3-carbonitrile, and pharmaceutically acceptable salts thereof; as well as: 3-[(S)-(4-chlorophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-chlorophenyl)methyl]benzonitrile, 3-[(S)-(4-cyanophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-cyanophenyl)methyl]benzonitrile, 3-[(S)-(4-cyanophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,2,4-oxadiazol-3-yl)phenyl]-2-methylpropyl}azetidin-1-yl)methyl]benzonitrile, 3-[(1S)-1-(1-{(S)-(4-cyanophenyl)[3-(1,2,4-oxadiazol-3-yl)phenyl]-methyl}azetidin-3-yl)-2-fluoro-2-methylpropyl]-5-fluorobenzonitrile, 5-(3-{1-[1-(diphenylmethyl)azetidin-3-yl]-2-fluoro-2-methylpropyl}-5-fluorophenyl)-1H-tetrazole, 5-(3-{1-[1-(diphenylmethyl)azetidin-3-yl]-2-fluoro-2-methylpropyl}-5-fluorophenyl)-1-methyl-1H-tetrazole, 5-(3-{1-[1-(diphenylmethyl)azetidin-3-yl]-2-fluoro-2-methylpropyl}-5-fluorophenyl)-2-methyl-2H-tetrazole, 3-[(4-chlorophenyl)(3-{2-fluoro-1-[3-fluoro-5-(2-methyl-2H-tetrazol-5-yl)phenyl]-2-methylpropyl}azetidin-1-yl)methyl]benzonitrile, 3-[(4-chlorophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(2-methyl-2H-tetrazol-5-yl)phenyl]-2-methylpropyl}azetidin-1-yl)methyl]benzonitrile, 5-{3-[(S)-{3-[(1S)-1-(3-bromo-5-fluorophenyl)-2-fluoro-2-methylpropyl]azetidin-1-yl}(4-chlorophenyl)methyl]phenyl}-1,3,4-oxadiazol-2(3H)-one, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl) [3-(1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,3,4-oxadiazol-2-yl)phenyl]methyl}azetidin-3-yl)-2-fluoro-2-methylpropyl]-5-fluorobenzonitrile, 3-((1S)-1-{1-[(S)-[3-(5-amino-1,3,4-oxadiazol-2-yl)phenyl] (4-chlorophenyl)methyl]azetidin-3-yl}-2-fluoro-2-methylpropyl)-5-fluorobenzonitrile, 3-((1S)-1-{1-[(S)-[3-(5-amino-1,3,4-oxadiazol-2-yl)phenyl](4-cyanophenyl)methyl]azetidin-3-yl}-2-fluoro-2-methylpropyl)-5-fluorobenzonitrile, 3-[(1S)-1-(1-{(S)-(4-cyanophenyl)[3-(1,2,4-oxadiazol-3-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,2,4-oxadiazol-3-yl)phenyl]methyl}azetidin-3-yl)-2-fluoro-2-methylpropyl]-5-fluorobenzonitrile, 5-[3-((S)-(4-chlorophenyl) {3-[(11S)-1-(3,5-difluorophenyl)-2-fluoro-2-methylpropyl]azetidin-1-yl}methyl)phenyl]-1,3,4-oxadiazol-2(3H)-one, 5-[3-((S)-(4-chlorophenyl) {3-[(1S)-1-(3,5-difluorophenyl)-2-fluoro-2-methylpropyl]azetidin-1-yl}methyl)phenyl]-1,3,4-oxadiazol-2(3H)-one, 4-{(S)-{3-[(1S)-1-(3,5-difluorophenyl)-2-fluoro-2-methylpropyl]azetidin-1-yl}[3-(5-oxo-4,5-dihydro-1,3,4-oxadiazol-2-yl)phenyl]methyl}-benzonitrile, ACOMPLIA (rimonabant, N-(1-piperidinyl)-5-(4-chlorophenyl)-1-(2,4-dichlorophenyl)-4-methylpyrazole-3-carboxamide, SR141716A), 3-(4-chlorophenyl-N′-(4-chlorophenyl)sulfonyl-N-methyl-4-phenyl-4,5-dihydro-1H-pyrazole-1-carboxamide (SLV-319), taranabant, N-[(1S,2S)-3-(4-Chlorophenyl)-2-(3-cyanophenyl)-1-methylpropyl]-2-methyl-2-[[5-(trifluoromethyl)-2-pyridinyl]oxy]propanamide, and pharmaceutically acceptable salts thereof.

Specific NPY5 antagonists that can be used in combination with the Angptl6 peptide compounds include: 3-oxo-N-(5-phenyl-2-pyrazinyl)-spiro[isobenzofuran-1(3H), 4′-piperidine]-1′-carboxamide, 3-oxo-N-(7-trifluoromethylpyrido[3,2-b]pyridin-2-yl)spiro-[isobenzofuran-1 (3H), 4′-piperidine]-1′-carboxamide, N-[5-(3-fluorophenyl)-2-pyrimidinyl]-3-oxospiro-[isobenzofuran-1 (3H), 4′-piperidine]-1′-carboxamide, trans-3′-oxo-N-(5-phenyl-2-pyrimidinyl)spiro[cyclohexane-1,1′(3′H)-isobenzofuran]-4-carboxamide, trans-3′-oxo-N-[1-(3-quinolyl)-4-imidazolyl]spiro[cyclohexane-1,1′(3′H)-isobenzofuran]-4-carboxamide, trans-3-oxo-N-(5-phenyl-2-pyrazinyl)spiro[4-azaiso-benzofuran-1(3H), 1′-cyclohexane]-4′-carboxamide, trans-N-[5-(3-fluorophenyl)-2-pyrimidinyl]-3-oxospiro[5-azaisobenzofuran-1(3H), 1′-cyclohexane]-4′-carboxamide, trans-N-[5-(2-fluorophenyl)-2-pyrimidinyl]-3-oxospiro[5-azaisobenzofuran-1 (3H), 1′-cyclohexane]-4′-carboxamide, trans-N-[1-(3,5-difluorophenyl)-4-imidazolyl]-3-oxospiro[7-azaisobenzofuran-1(3H), 1′-cyclohexane]-4′-carboxamide, trans-3-oxo-N-(1-phenyl-4-pyrazolyl)spiro[4-azaisobenzofuran-1(3H), 1′-cyclohexane]-4′-carboxamide, trans-N-[1-(2-fluorophenyl)-3-pyrazolyl]-3-oxospiro[6-azaisobenzofuran-1(3H), 1′-cyclohexane]-4′-carboxamide, trans-3-oxo-N-(1-phenyl-3-pyrazolyl)spiro[6-azaisobenzofuran-1(3H), 1′-cyclohexane]-4′-carboxamide, trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof.

Specific ACC-1/2 inhibitors that can be used in combination with the Angptl6 peptide compounds include: 1′-[(4,8-dimethoxyquinolin-2-yl)carbonyl]-6-(1H-tetrazol-5-yl)spiro[chroman-2,4′-piperidin]-4-one; (5-{1′-[(4,8-dimethoxyquinolin-2-yl)carbonyl]-4-oxospiro[chroman-2,4′-piperidin]-6-yl}-2H-tetrazol-2-yl)methyl pivalate; 5-{1′-[(8-cyclopropyl-4-methoxyquinolin-2-yl)carbonyl]-4-oxospiro[chroman-2,4′-piperidin]-6-yl}nicotinic acid; 1′-(8-methoxy-4-morpholin-4-yl-2-naphthoyl)-6-(1H-tetrazol-5-yl)spiro[chroman-2,4′-piperidin]-4-one; and 1′-[(4-ethoxy-8-ethylquinolin-2-yl)carbonyl]-6-(1H-tetrazol-5-yl)spiro[chroman-2,4′-piperidin]-4-one; and pharmaceutically acceptable salts and esters thereof. MK-3887, L-001738791.

Specific MCH1R antagonist compounds that can be used in combination with the Angptl6 peptide compounds include: 1-{4-[(1-ethylazetidin-3-yl)oxy]phenyl}-4-[(4-fluorobenzyl)oxy]pyridin-2(1H)-one, 4-[(4-fluorobenzyl)oxy]-1-{4-[(1-isopropylazetidin-3-yl)oxy]phenyl}pyridin-2(1H)-one, 1-[4-(azetidin-3-yloxy)phenyl]-4-[(5-chloropyridin-2-yl)methoxy]pyridin-2(1H)-one, 4-[(5-chloropyridin-2-yl)methoxy]-1-{4-[(1-ethylazetidin-3-yl)oxy]phenyl}pyridin-2(1H)-one, 4-[(5-chloropyridin-2-yl)methoxy]-1-{4-[(1-propylazetidin-3-yl)oxy]phenyl}pyridin-2(1H)-one, and 4-[(5-chloropyridin-2-yl)methoxy]-1-(4-{[(2S)-1-ethylazetidin-2-yl]methoxy}phenyl)pyridin-2(1H)-one, or a pharmaceutically acceptable salt thereof.

A specific DP-IV inhibitor that can be used in combination with the Angptl6 peptide compounds is 7-[(3R)-3-amino-4-(2,4,5-trifluorophenyl)butanoyl]-3-(trifluoromethyl)-5,6,7,8-tetrahydro-1,2,4-triazolo[4,3-a]pyrazine, or a pharmaceutically acceptable salt thereof.

Specific H3 (histamine H3) antagonists/inverse agonists that can be used in combination with the Angptl6 peptide compounds include: those described in WO05/077905, including: 3-{4-[(1-cyclobutyl-4-piperidinyl)oxy]phenyl}-2-ethylpyrido[2,3-d]-pyrimidin-4(3H)-one, 3-{4-[(1-cyclobutyl-4-piperidinyl)oxy]phenyl}-2-methylpyrido[4,3-d]pyrimidin-4(3H)-one, 2-ethyl-3-(4-{3-[(3S)-3-methylpiperidin-1-yl]propoxy}phenyl)pyrido[2,3-d]pyrimidin-4(3H)-one 2-methyl-3-(4-{3-[(3S)-3-methylpiperidin-1-yl]propoxy}phenyl)pyrido[4,3-d]pyrimidin-4(3H)-one, 3-{4-[(1-cyclobutyl-4-piperidinyl)oxy]phenyl}-2,5-dimethyl-4(3H)-quinazolinone, 3-{4-[(1-cyclobutyl-4-piperidinyl)oxy]phenyl}-2-methyl-5-trifluoromethyl-4(3H)-quinazolinone, 3-{4-[(1-cyclobutyl-4-piperidinyl)oxy]phenyl}-5-methoxy-2-methyl-4(3H)-quinazolinone, 3-{4-[(1-cyclobutylpiperidin-4-yl)oxy]phenyl}-5-fluoro-2-methyl-4(3H)-quinazolinone, 3-{4-[(1-cyclobutylpiperidin-4-yl)oxy]phenyl}-7-fluoro-2-methyl-4(3H)-quinazolinone, 3-{4-[(1-cyclobutylpiperidin-4-yl)oxy]phenyl}-6-methoxy-2-methyl-4(3H)-quinazolinone, 3-{4-[(1-cyclobutylpiperidin-4-yl)oxy]phenyl}-6-fluoro-2-methyl-4(3H)-quinazolinone, 3-{4-[(1-cyclobutylpiperidin-4-yl)oxy]phenyl}-8-fluoro-2-methyl-4(3H)-quinazolinone, 3-{4-[(1-cyclopentyl-4-piperidinyl)oxy]phenyl}-2-methylpyrido[4,3-d]pyrimidin-4(3H)-one, 3-{4-[(1-cyclobutylpiperidin-4-yl)oxy]phenyl}-6-fluoro-2-methylpyrido[3,4-d]pyrimidin-4(3H)-one, 3-{4-[(1-cyclobutyl-4-piperidinyl)oxy]phenyl}-2-ethylpyrido[4,3-d]pyrimidin-4(3H)-one, 6-methoxy-2-methyl-3-{4-[3-(1-piperidinyl)propoxy]phenyl}pyrido[3,4-d]pyrimidin-4(3H)-one, 6-methoxy-2-methyl-3-{4-[3-(1-pyrrolidinyl)propoxy]phenyl}pyrido[3,4-d]pyrimidin-4(3H)-one, 2,5-dimethyl-3-{4-[3-(1-pyrrolidinyl)propoxy]phenyl}-4(3H)-quinazolinone, 2-methyl-3-{4-[3-(1-pyrrolidinyl)propoxy]phenyl}-5-trifluoromethyl-4(3H)-quinazolinone, 5-fluoro-2-methyl-3-{4-[3-(1-piperidinyl)propoxy]phenyl}-4(3H)-quinazolinone, 6-methoxy-2-methyl-3-{4-[3-(1-piperidinyl)propoxy]phenyl}-4(3H)-quinazolinone, 5-methoxy-2-methyl-3-(4-{3-[(3S)-3-methylpiperidin-1-yl]propoxy}phenyl)-4(3H)-quinazolinone, 7-methoxy-2-methyl-3-(4-{3-[(3S)-3-methylpiperidin-1-yl]propoxy}phenyl)-4(3H)-quinazolinone, 2-methyl-3-(4-{3-[(3S)-3-methylpiperidin-1-yl]propoxy}phenyl)pyrido[2,3-d]pyrimidin-4(3H)-one, 5-fluoro-2-methyl-3-(4-{3-[(2R)-2-methylpyrrolidin-1-yl]propoxy}phenyl)-4(3H)-quinazolinone, 2-methyl-3-(4-{3-[(2R)-2-methylpyrrolidin-1-yl]propoxy}phenyl)pyrido[4,3-d]pyrimidin-4(3H)-one, 6-methoxy-2-methyl-3-(4-{3-[(2R)-2-methylpyrrolidin-1-yl]propoxy}phenyl)-4(3H)-quinazolinone, 6-methoxy-2-methyl-3-(4-{3-[(2S)-2-methylpyrrolidin-1-yl]propoxy}phenyl)-4(3H)-quinazolinone, and pharmaceutically acceptable salts thereof.

Specific CCK1R agonists of use in combination with the Angtl6 peptide compounds include: 3-(4-{[1-(3-ethoxyphenyl)-2-(4-methylphenyl)-1H-imidazol-4-yl]carbonyl}-1-piperazinyl)-1-naphthoic acid; 3-(4-{[1-(3-ethoxyphenyl)-2-(2-fluoro-4-methylphenyl)-1H-imidazol-4-yl]carbonyl}-1-piperazinyl)-1-naphthoic acid; 3-(4-{[1-(3-ethoxyphenyl)-2-(4-fluorophenyl)-1H-imidazol-4-yl]carbonyl}-1-piperazinyl)-1-naphthoic acid; 3-(4-{[1-(3-ethoxyphenyl)-2-(2,4-difluorophenyl)-1H-imidazol-4-yl]carbonyl}-1-piperazinyl)-1-naphthoic acid; and 3-(4-{[1-(2,3-dihydro-1,4-benzodioxin-6-yl)-2-(4-fluorophenyl)-1H-imidazol-4-yl]carbonyl}-1-piperazinyl)-1-naphthoic acid; and pharmaceutically acceptable salts thereof. MK-8406

Specific MC4R agonists of use in combination with the Angtl6 peptide compounds include: 1) (5S)-1′-{[(3R,4R)-1-tert-butyl-3-(2,3,4-trifluorophenyl)piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 2) (5R)-1′-{[(3R,4R)-1-tert-butyl-3-(2,3,4-trifluorophenyl)-piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 3) 2-(1′-{[(3S,4R)-1-tert-butyl-4-(2,4-difluorophenyl)pyrrolidin-3-yl]carbonyl}-3-chloro-2-methyl-5H-spiro[furo[3,4-b]pyridine-7,4′-piperidin]-5-yl)-2-methylpropanenitrile; 4) 1′-{[(3S,4R)-1-tert-butyl-4-(2,4-difluorophenyl)pyrrolidin-3-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 5) N-[(3R,4R)-3-({3-chloro-2-methyl-5-[1-methyl-1-(1-methyl-1H-1,2,4-triazol-5-yl)ethyl]-1′H,5H-spiro[furo-[3,4-b]pyridine-7,4′-piperidin]-1′-yl}carbonyl)-4-(2,4-difluorophenyl)-cyclopentyl]-N-methyltetrahydro-2H-pyran-4-amine; 6) 2-[3-chloro-1′-({(1R,2R)-2-(2,4-difluorophenyl)-4-[methyl(tetrahydro-2H-pyran-4-yl)amino]-cyclopentyl}-carbonyl)-2-methyl-5H-spiro[furo[3,4-b]pyridine-7,4′-piperidin]-5-yl]-2-methyl-propane-nitrile; and pharmaceutically acceptable salts thereof.

Additionally, other peptide analogs and mimetics of the incretin hormone glucagon-like peptide 1(GLP-1), may also be of use in combination with the Angtl6 peptide compounds.

Methods of administrating the pharmacological compositions comprising the one or more Angtl6 peptide compounds to an individual include, but are not limited to, intradermal, intramuscular, intraperitoneal, intravenous, subcutaneous, intranasal, epidural, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Intraventricular injection may be facilitated by an intraventricular catheter attached to a reservoir (for example, an Ommaya reservoir). Pulmonary administration may also be employed by use of an inhaler or nebulizer, and formulation with an aerosolizing agent. It may also be desirable to administer the one or more Angtl6 peptide compounds locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the Angptl6 peptide compounds including, but not limited to, encapsulation in liposomes, microparticles, microcapsules; minicells; polymers; capsules; tablets; and the like. In one embodiment, the Angtl6 peptide compounds may be delivered in a vesicle, in particular a liposome. In a liposome, the Angtl6 peptide compound is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028 and U.S. Pat. No. 4,737,323. In yet another embodiment, the Angtl6 peptide compound can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (for example, the brain), thus requiring only a fraction of the systemic dose. See, for example, Goodson, In: Medical Applications of Controlled Release, 1984. (CRC Press, Bocca Raton, Fla.).

The amount of the compositions comprising the one or more Angtl6 peptide compounds which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the composition with which to treat each individual patient. Initially, the attending physician will administer low doses of the composition and observe the patient's response. Larger doses of the composition may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. However, suitable dosage ranges for intravenous administration of the compositions comprising the Angptl6 peptide are generally about 5-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions comprising one or more of the Angtl6 peptide compounds disclosed herein. Dosage will also vary according to the age, weight and response of the individual patient.

Further provided is a pharmaceutical pack or kit, comprising one or more containers filled with one or more of the ingredients of the pharmaceutical compositions and Angptl6 peptide compounds. Optionally associated with such container(s) may be a notice in the form prescribed by a governmental agency regulating the manufacture, use or sale of pharmaceuticals or biological products, which notice reflects approval by the agency of manufacture, use or sale for human administration.

The following examples are intended to promote a further understanding of the present invention.

### EXAMPLE 1

In this example, adenovirus (Ad) overexpressing full-length Angptl6 or N-terminus portion of the protein (containing the coiled-coil domain) were constructed and tested in vivo.

### Recombinant Adenovirus Preparation:

Angptl6 full-length protein (Angptl6) and the N-terminus Angplt6 (NAngptl6) peptide were PCR amplified using the full-length cDNA encoding Angptl6 (Invitrogen) as template. PCR fragments were sub-cloned into the Gateway entry vector pENTR1A (Invitrogen) containing the CMV promoter to generate Pterm-Angptl6 and Pterm-NAngptl6 clones. These PCR primers were used to generate a DNA encoding the full-length Angptl6 protein: FORWARD: TCAGGATCCGTGGGATTGCCGCAAACCTC (SEQ ID NO:11); REVERSE: AGCTGAAGGAGATAGGAACA (SEQ ID NO:12). These PCR primers were used to generate DNA encoding the NAngptl6 peptide: FORWARD: TCAGGATCCGTGGGATTGCCGCAAACCTC (SEQ ID NO:13) and REVERSE: GGTGCTCGAGTCAAGAAGATGGAGGCCCCTGCTG (SEQ ID NO:14).

In order to generate the recombinant adenovirus vectors expressing full-length Angplt6 protein and NAngplt6 peptide, expression cassettes prepared above were recombined into Gateway-based pAd-Block-iT DEST vector (Invitrogen) to make Ad-Angptl6 and Ad-NAngptl6, respectively. Recombinant adenoviruses were produced in HEK293 cells and purified by two rounds of CsCl density gradient ultracentrifugation. The purified virus was de-salted by dialysis and concentrated over CentriPrep YM-50 column before use. The expression of full-length or N-terminus Angptl6 in vitro was confirmed by real time PCR.

### Analysis of Over Expression of Angptl6 Protein in Diet Induced Obese Mice.

To phenotype the diabetes and obesity traits associated with Angptl6 protein and NAngptl6 peptide administered to diabetic or obese mice, two sequential experiments were performed in an established diet induced obese (DIO) mouse model.

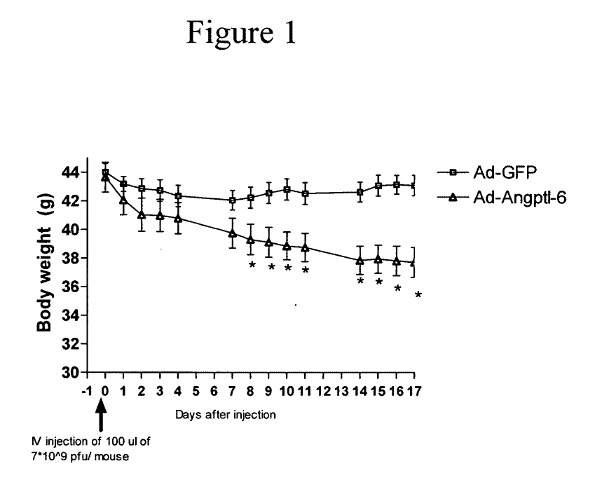

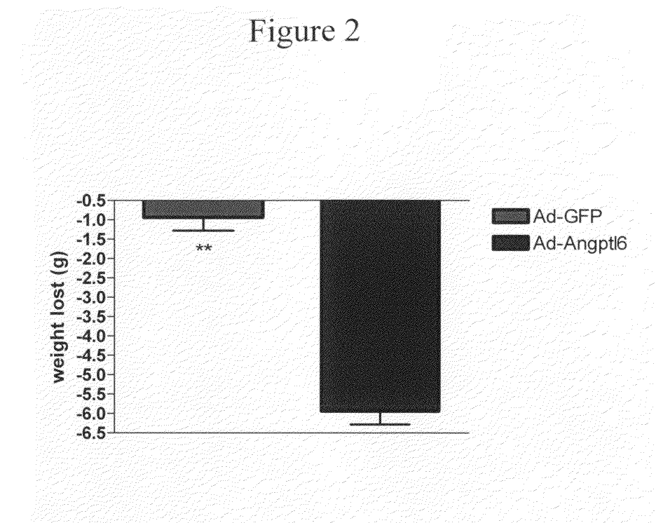

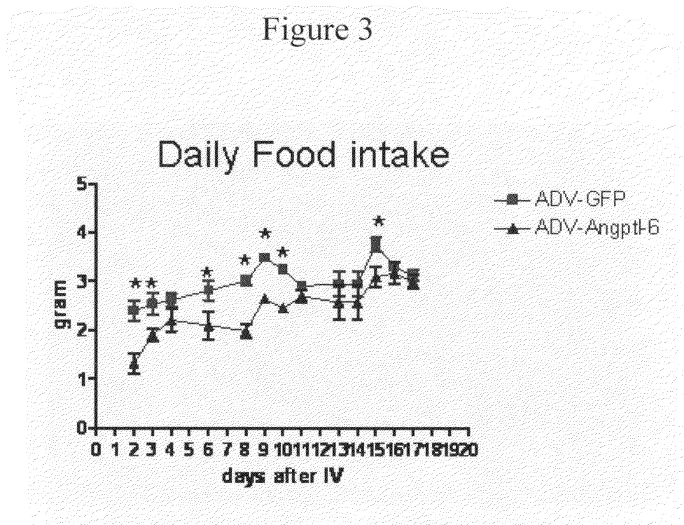

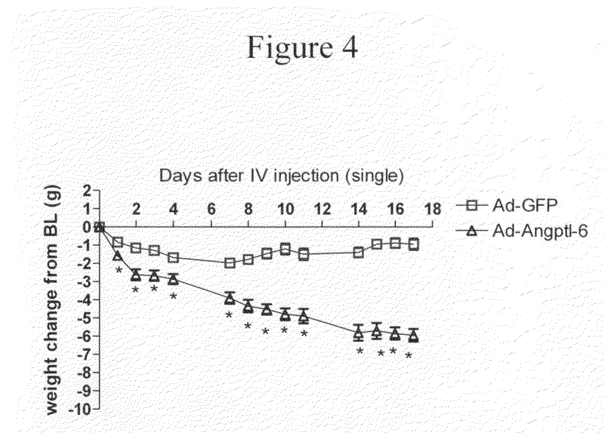

Mice were monitored for food intake (FI) and body weight (BW) two weeks prior to the experiment and were divided into separate cohorts such that their BW and feeding behaviors were similar. These cohorts were treated with intravenous (IV) delivery of either Ad-Angptl6 or Ad-GFP (control that expresses green fluorescent protein). Virally treated groups showed a significant reduction in overnight BW gain and FI relative to saline treated mice (FIGS. 1, 3, and 4). This is a phenomenon that is often observed and it is attributed to an immune response associated with the introduction of adenovirus. Ad-GFP injected mice re-bounded in terms of FI following the first week of treatment. However mice treated with the Ad-Angptl6 continued to lose weight throughout the study (FIGS. 1 and 4). Food intake in the Ad-Angptl6 treated group was significantly reduced the first 10 days of the experiments, however, at the last seven days of the treatment, we did not detect a significant effect on FI although BW continued to be reduced (FIG. 3). At 17 days after treatment, Ad-Angptl6 treated mice lost 12% their BW respectively relative to the Ad-GFP treated mice (FIG. 2). NMR analysis performed pre- and post-treatment revealed that the reduction in BW in Ad-Angptl6 was due primarily to fat-mass loss compared to the Ad-GFP with minimal effect on muscle mass. Consistent with these observations, leptin levels were significantly reduced in mice treated with Ad-Angptl6 compared to Ad-GFP. Furthermore, fed glucose and insulin levels were also significantly reduced.

### Analysis of Over Expression of Angptl6 Protein and NAngptl6 Peptide in Diet Induced Obese Mice

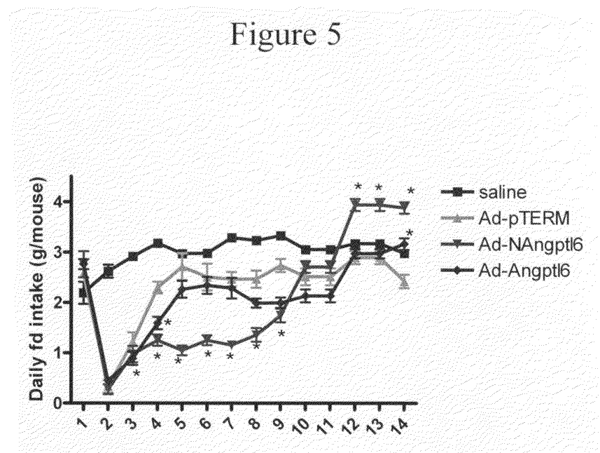

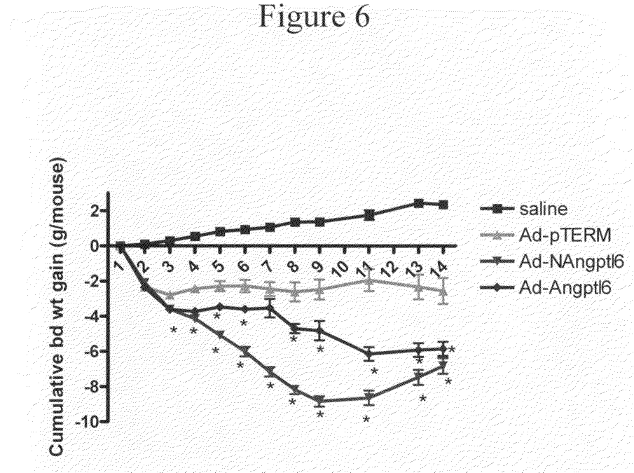

A similar study to the above was performed on four separate cohorts of DIO mice. In this study, the cohorts were treated with either saline, empty virus control (Ad-Pterm), Ad-Angptl6, or Ad-NAngptl6. DIO mice treated with full length Ad-Angptl6, Ad-NAngptl6, Ad-Pterm, or saline were monitored for food intake and body weight for two wks. As previously observed, Ad expressing full-length Angptl6 protein lost significant weight relative to the control treated mice. However, Ad expressing NAngptl6 peptide showed much greater efficacy in terms of weight loss relative to the Ad expressing the full-length Angptl6 protein (FIG. 6). Furthermore, a significant reduction in daily food intake was observed in these mice relative to the mice treated with Ad expressing Angptl6 protein (FIG. 5).

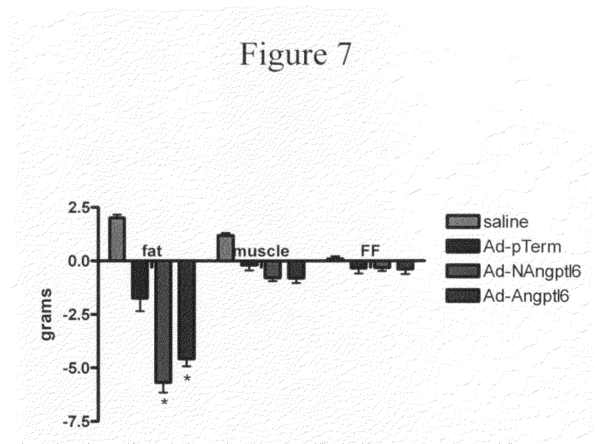

At eight days after delivery, we observed 19% reduction in BW in mice treated with Ad-NAngptl6 relative to the control treated mice. However, these mice rebounded in terms of BW and by the end of the two-week study had lost weight similar to the Angptl6 treated mice (FIG. 6). This might be due to the fact because Ad is transient, NAngptl6 expression was reduced after the first wk relative to the Ad-Angptl6 treated mice. This is consistent with hepatic mRNA levels in these two groups. FIG. 7 shows the weight change in fat, muscle, and free fluid (FF) in mice administered either a single IV dose of saline, control vector (Ad-pterm), adenovirus-mouse angptl6 (Ad-Angptl6), or adenovirus-N-terminal mouse Angtpl6 (Ad-NAngptl6).

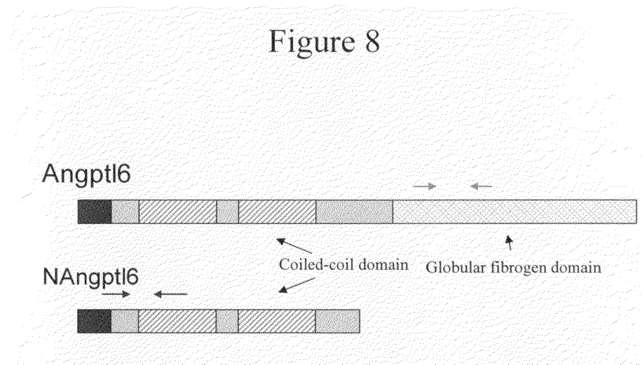

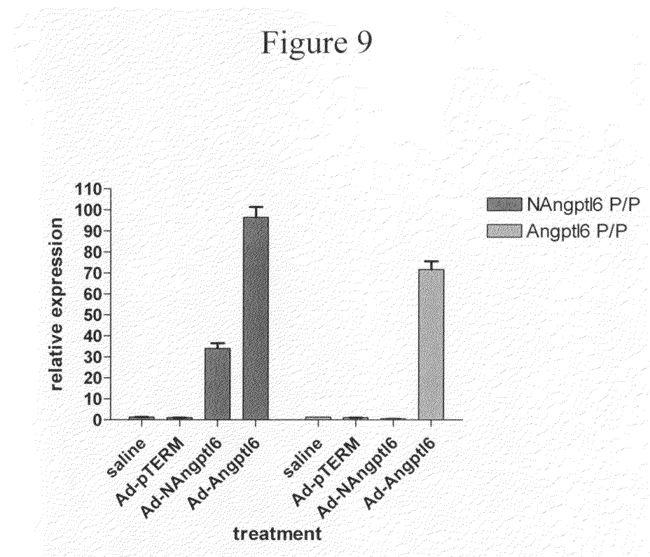

Hepatic mRNA levels of Angptl6, as well as NAngptl6 mRNA levels were measured at two weeks after virus delivery. We observed approximately a 70-fold increase of full-length Angptl6 expression relative to endogenous levels in mice treated with Ad-NAngptl6 relative to the control virally infected group but only a 30-fold increase in NAngptl6 was detected in the mice treated with Ad-NAngptl6 (FIG. 9). FIG. 8 is schematic showing the position of PCR primers used to detect expression of mouse angptl6 (Adv-Angptl6) or the N-terminal mouse Angtpl6 (Ad-NAngptl6).

In conclusion, adenovirus vectors expressing N-terminal truncated Angptl6 peptide showed much greater efficacy in terms of weight loss relative to the Adenovirus expressing the full-length Angptl6 protein. Furthermore, a significant reduction in daily food intake was observed in these mice relative to the mice treated with Adenovirus expressing full-length Angptl6 protein. Hepatic mRNA levels of Angptl6, as well as truncated Angptl6 mRNA levels, were significantly elevated two weeks after delivery. These data indicate that the coiled-coil portion of the Angptl6 protein is sufficient to achieve the metabolic correction previously observed with the full length protein. Thus, derivatives of Angptl6 may be novel therapeutics for the treatment of obesity and diabetes.

### Total RNA Isolation and Real-Time Quantitative PCR Analysis:

Frozen liver samples were homogenized with a Polytron in Trizol reagent (Invitrogen, Carlsbad, Calif.). Total RNA was purified using Qiagen RNeasy kit (Valencia, Calif.). cDNA was synthesized by using Qiagen OmniScript RT kit (Valencia, Calif.) with random hexamers. Real-Time quantitative PCR measurements were performed with Roche LightCycler 480 Instrument (Roche Applied Science, Indianapolis, Ind.). Angptl6 primer-probe sets were purchased as an Assay-on-Demand kit from Applied Biosystems (Foster city, CA). Angptl 6-Nterm primer-probe were custom designed. The relative quantification for a given gene was corrected to 18S mRNA levels. FIG. 8 is schematic showing the position of PCR primers used to detect expression of mouse angptl6 (Adv-Angptl6) or the N-terminal mouse Angtpl6 (Ad-NAngptl6).

### Animals and Diets.

All animal protocols used in these studies were approved by the Merck Research Laboratories Institutional Animal Care and Use Committee in Rahway, N.J. Four months old diet-induced obese C57/BL6 male mice (Taconic Farm, Germantown, N.Y.) were individually housed with ad libitum access to food and water in a 12-hour/12-hour light/dark cycle. These mice were fed with high fat diet [HF, D12492i: 60% Kcal from fat, 20% Kcal from carbohydrate, 20% Kcal from protein, 5.2 kcal/g (Research Diets, New Brunswick, N.J.)]. When mice reached (about 42 g) they were split into cohorts (n=8/group) with similar body weights and feeding behaviors. Mice were injected with 100 uL of Ad containing 5×10⁹ particles of either Ad-GFP, Ad-Pterm, Ad-Angptl6, or Ad-Angptl-Nterm. Body weight and food intake measurements were taken daily at the same time of the day. At the end of the study, mice were anesthetized with isoflurane. Blood was collected by cardiac puncture. Middle liver lobe was collected from each mouse and was snap frozen in liquid nitrogen. A section of the liver was postfixed in Prefer solution (Anatech LTD, Battle Creek, Mich., USA) and paraffin embedded for subsequent pathology analysis.

### EXAMPLE 2

The N-terminal domain of Antptl6 can also be fused at either end to a peptide tag such as a Flag tag or hexahistidine tag to aid in purification and detection of the recombinant protein. The protein can be expressed in E. coli, yeast (such as Pichia pastoris or Saccharomyces cerevisiae), or mammalian cells.

A fusion protein can also be made with mouse or human Angtpl6 peptide fragments and the Fc region of human or mouse IgG top be expressed in mammalian cells. Such a fusion will extend the serum half life of the administered protein. The fusion may be placed at the N or C terminal of the N-terminal Angptl6 peptide and may contain a linker or “hinge” amino acid sequence. For human Angptl6, the N-terminal Angptl6 domain contains either 1-240 or 1-217 amino acids; for mouse Angptl6, 1-227, or 1-204 or 25-227. The Fc moiety can be derived from mouse IgG₁ or human IgG₂M4. The secretive leader sequence can be the original (in the case of those constructs that start with amino acid 1) or from another protein (in the case of 25-227). The linker regions between the Angptl6 domain and the Fc domain contain one or both of GGG and the hinge region. The hinge region can be partial or full-length. The N-terminal domain and full-length Angptl6 were also tagged with hexahistidine for the expression in mammalian cells. The sequences for all the constructs are listed below in Tables 1 and 2. SEQ ID NOs: 21, 30-32 show constructs in which the endogenous leader is replaced with an IgG leader.

TABLE 1

| | Secretion | Angptl6 N- | | | |
|---|---|---|---|---|---|
| Angptl6 | Leader | terminal | | IgG FC | |
| Origin | Sequence | Domain | Linker | Domain | SEQ ID NO. |
| Mouse | Mouse IgG | 25-227 | Full-length Mouse | Mouse IgG1 | 21 |
| | | | IgG1 Hinge Region | FC Domain | |
| | Angptl6 | 1-227 | Full-length Mouse | Mouse IgG1 | 22 |
| | | | IgG1 Hinge Region | FC Domain | |
| | Angptl6 | 1-204 | Full-length Mouse | Mouse IgG1 | 23 |
| | | | IgG1 Hinge Region | FC Domain | |
| Human | Angptl6 | 1-240 | Full-length Mouse | Mouse IgG1 | 24 |
| | | | IgG1 Hinge Region | FC Domain | |
| | Angptl6 | 1-240 | Full-length Human | Human | 25 |
| | | | IgG2M4 Hinge | IgG2M4 FC | |
| | | | Region | Domain | |
| | Angptl6 | 1-240 | Partial human IgG2 | Human | 26 |
| | | | Hinge Region | IgG2M4 FC | |
| | | | | Domain | |
| | Angptl6 | 1-240 | GGG + Full-length | Human | 27 |
| | | | human IgG2 Hinge | IgG2M4 FC | |
| | | | Region | Domain | |
| | Angptl6 | 1-240 | GGG + Partial human | Human | 28 |
| | | | IgG2 Hinge Region | IgG2M4 FC | |
| | | | | Domain | |
| | Angptl6 | 1-217 | Full-length human | Human | 29 |
| | | | IgG2 Hinge Region | IgG2M4 FC | |
| | | | | Domain | |

TABLE 2

| | Secretion | | | | |
|---|---|---|---|---|---|
| Angptl6 | Leader | N-terminal | Angptl6 | C-terminal | |
| Origin | Sequence | Tag | Domain | Tag | SEQ ID NO: |
| Mouse | Mouse IgG | FLAG | 25-227 | 6His | 30 |
| | Mouse IgG | FLAG | 25-457 | 6His | 31 |
| Human | Mouse IgG | FLAG | 25-470 | 6His | 32 |

The Angptl6 peptide-Fc fusions were designed with the strategy outlined above and the corresponding DNAs were chemically synthesized with flanking sequences and cloned into expression vectors using PstI and NotI sites. The expression vector contains human cytomegalovirus early promoter and bovine growth hormone polyadenylation signal. The PstI-NotI fragment contains Kozak sequences in front of the translation initiation start codon.

The expression vectors carry oriP from EBV viral genome for prolonged expression in 293EBNA cells and the bacterial sequences for kanamycin selection marker and replication origin in E. coli. The antibodies were expressed in 293 suspension cells. The plasmids were transfected using PEI based transfection reagents. The transfected cells were incubated in Opti-MEM serum free medium and the secreted ANgptl6 peptide-Fc fusion proteins were purified from medium using protein A/G affinity chromatography. The concentration of purified antibodies was determined by OD280 nm and the purity by LabChip capillary electrophoresis. For hexahistidine tagged proteins, an IMAC based chromatograph is used according to manufacturer's recommendation.

### EXAMPLE 3

A DNA sequence (SEQ ID NO:7) encoding a mouse Angtl6 peptide fusion protein with a hexahistine tag at the N-terminus may be prepared by PCR amplification of mouse angptl6 cDNA obtained from a commercial vendor using primers with Nde1 (SEQ ID NO:8) and Xho1 (SEQ ID NO:9) restriction sites attached. The DNA is cut with Nde1 and Xho1 and ligated into plasmid pET28b (Novagen) such that the expressed Angptl6 peptide fusion protein had the amino acid sequence shown in SEQ ID NO:10, including a N-Terminal histidine tag.

An E. coli strain such as BL21 (DE3) pLysS is transformed with the plasmid using standard methods. The transformed E. coli are grown in Terrific Broth (Teknova) at 37° C. to an optical density between 0.6 and 1.0 at 600 nm and then induced with IPTG. The cells are allowed to grow for three more hours and then harvested by centrifugation. The cells are lysed by three freeze thaw cycles followed by the addition of lysozyme (60,000 units/gram of cells, Epicentre Biotechnologies) and endonuclease (1,000 units/gram of cells, Epicentre Biotechnologies), incubated for 15 minutes at 37° C. and centrifuged at 27000×g for 20 minutes at 4° C. The supernatant is applied to a Ni affinity column and eluted with imidazole as described by the manufacture (Novagen). Alternatively, the protein may be expressed as insoluble inclusion bodies. In this case, the Angtl6 peptide fusion protein is solubilized and purified in the presence of 6M urea. The urea can then be removed by dialysis. The Angptl6 peptide fusion protein is first reduced with 10 mM DTT for ten minutes at room temperature and then exchanged into a buffer such as 0.75 M guanidine HCl. 0.25M NaCl, 1 mM DTT, 1 mM EDTA, and 50 mM Tris pH 8.0. The Angptl6 peptide fusion protein can then be dialyzed into a buffer consisting of 0.75 M arginine and 0.25 M NaCl. The refolded Angptl6 peptide fusion protein in this buffer can then be administered to mice by a subcutaneous pump.

A His tag Angptl6 peptide fusion protein can also be made with the human protein. The DNA can be obtained from PCR of a human cDNA library or synthesized as shown in SEQ ID No:15 and used as above to obtain the Angptl6 peptide fusion protein with the amino acid shown in SEQ ID No:16.

1. A compound for treatment of obesity or diabetes comprising:
an angiopoietin-like protein 6 (Angptl6) peptide comprising the coiled-coil domain of an Angptl6 protein and excluding an intact globular fibrinogen domain of the Angptl6 protein.

2. The compound of claim 1, wherein the Angptl6 peptide comprises an amino acid sequence with at least 95% identity to the amino acid sequence set forth in SEQ ID NO:1.

3. The compound of claim 1 wherein the peptide is conjugated to a heterologous protein or peptide.

4. The compound of claim 7 wherein the heterologous protein is selected from the group consisting of human serum albumin, immunoglobulin, and transferrin.

5. The compound of claim 1 wherein the compound comprises a fusion protein comprising the Angptl6 peptide fused to a heterologous protein or peptide.

6. The compound of claim 5, wherein the heterologous protein is the Fc domain of an immunoglobulin.

7. A The compound of claim 1 comprising the formula
Z¹-peptide-Z²
wherein the peptide is the Angptl6 peptide comprising the coiled-coil domain of an Angptl6 protein and excluding an intact globular fibrinogen domain of the Angptl6 protein, wherein one or more of amino acids of the peptide can be a D- or L-amino acid, an amino acid analog, or an amino acid derivative; Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof.

8. (canceled)

9. The compound of claim 7 wherein the N-terminal amino acid is covalently joined to one or more molecules selected from the group consisting of PEG, cholesterol, N-ethylmaleimidyl, and palmitoyl.

10. The compound of claim 7 wherein the peptide further includes a cysteine residue at the N-terminus of the peptide to which is optionally present a protecting group that, if present, is joined to the N-terminal amino group of the cysteine residue.

11. The compound of claim 10 wherein the thiol group of the cysteine residue at the N-terminus is covalently joined to one or more molecules selected from the group consisting of PEG, cholesterol, N-ethylmaleimidyl, and palmitoyl.

12. A method for treating a metabolic disorder in an individual comprising:
administering to the individual a therapeutically effective amount of an angiopoietin-like protein 6 (Angptl6) peptide comprising the coiled-coil domain of an Angptl6 protein and excluding an intact globular fibrinogen domain of the Angptl6 protein.

13. (canceled)

14. The method of claim 12 wherein the peptide is conjugated to a heterologous protein.

15. The method of claim 14 wherein the heterologous protein is selected from the group consisting of human serum albumin, immunoglobulin, transferrin.

16. The method of claim 12 wherein the compound comprises a fusion protein comprising the Angptl6 peptide fused to a heterologous protein or peptide.

17. The method of claim 16, wherein the heterologous protein is the Fc domain of an immunoglobulin.

18. The method of claim 12 wherein the metabolic disorder is selected from the group consisting of obesity, metabolic syndrome or syndrome X, type II diabetes, complications of diabetes, hypertension, dyslipidemias, cardiovascular disease, gallstones, osteoarthritis, insulin resistance, and certain forms of cancers.

19. The method of claim 12 wherein the angiopoietin-like protein 6 (Angptl6) peptide comprises the formula
Z¹-peptide-Z²
wherein the peptide is the Angptl6 peptide comprising the coiled-coil domain of an Angptl6 protein and excluding an intact globular fibrinogen domain of the Angptl6 protein, wherein one or more of amino acids of the peptide can be a D- or L-amino acid, an amino acid analog, or an amino acid derivative; Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof.

20. (canceled)

21. The method of claim 19 wherein the N-terminal amino acid is covalently joined to one or more molecules selected from the group consisting of PEG, cholesterol, N-ethylmaleimidyl, and palmitoyl.

22. The method of claim 19 wherein the peptide further includes a cysteine residue at the N-terminus of the peptide to which is optionally present a protecting group that, if present, is joined to the N-terminal amino group of the cysteine residue.

23. The method of claim 22 wherein the thiol group of the cysteine residue at the N-terminus is covalently joined to one or more molecules selected from the group consisting of PEG, cholesterol, N-ethylmaleimidyl, and palmitoyl.

24. (canceled)
